(19) United States
(12) Patent Application Publication
(10) Pub. No.: US 2019/0161516 A1
(43) Pub. Date: May 30, 2019

(54) PROTEASOME INHIBITORS

(71) Applicant: University of Kentucky Research Foundation, Lexington, KY (US)

(72) Inventor: Kyung Bo Kim, Lexington, KY (US)

(21) Appl. No.: 16/021,710

(22) Filed: Jun. 28, 2018

Related U.S. Application Data
(60) Provisional application No. 62/592,688, filed on Nov. 30, 2017.

(51) Int. Cl. C07K 5/10; A61P 25/28; A61P 27/02

(52) U.S. Cl. CPC C07K 5/10; A61K 38/00; A61P 27/02; A61P 25/28

(57) ABSTRACT

Unique epoxyketone compounds useful for inhibiting a proteasome in a cell, pharmaceutical compositions and methods of their use are provided herein.

### RELATED APPLICATIONS

This application claims priority from U.S. Provisional Application Ser. No. 62/592,688 filed Nov. 30, 2017, the entire disclosure of which is incorporated herein by this reference.

### GOVERNMENT INTEREST

This invention was made with government support under R01 CA188354 awarded by National Institutes of Health. The government has certain rights in the invention.

### TECHNICAL FIELD

The presently-disclosed subject matter relates to unique peptide epoxyketones, their pharmaceutically acceptable salts, process(es) for their preparation, pharmaceutical compositions containing the unique proteasome inhibitors, and methods of treating disease(s) in a subject, including cancer, via administration of the peptide epoxyketones.

### INTRODUCTION

The proteasome is a key player in one of the most fundamental processes in eukaryotic cells, the ubiquitin-dependent protein degradation pathway. The proteasome is a large multi-subunit protease that degrades the majority of cellular proteins. The proteasome also controls critical cellular process, such as the cell cycle, via the regulated degradation of signaling proteins. By perturbing these processes, inhibition of the proteasome leads to apoptosis, especially in cancer cells. It is for this reason that proteasome inhibitors have become critically important therapies in the treatment of multiple myeloma.

Although the distinct catalytic subunits of the different proteasome isoforms have been suggested to play roles in adding antigenic diversity to peptides generated from protein degradation, the catalytic subunits responsible for the CT-L activity (β5 and β5i) are thought to be most physiologically important and have been recognized as the key targets of bortezomib (Velcade®), the first-in-class proteasome inhibitor approved by the FDA in 2003 for the treatment of relapsed multiple myeloma (MM), and carfilzomib, a second-generation proteasome inhibitor approved by the FDA for the treatment of relapsed multiple myeloma patients who have received at least two prior therapies, including bortezomib. The addition of these proteasome inhibitors to chemotherapeutic armaments has dramatically improved the therapeutic landscape for patients with multiple myeloma (MM). Despite the remarkable successes of these drugs in the clinic, intrinsic and acquired drug resistance remains a major clinical challenge. Additionally, these drugs have failed to provide clinical benefit to patients with solid cancers, further adding to the need for next generation proteasome inhibitors.

Alzheimer's disease (AD) is the most common form of dementia and poses a great health-care challenge of the 21st century as aging population continues to grow. Intensive research efforts have been put forth for more than three decades, yet little is known about the cause of AD. The discoveries of amyloid β (Aβ) and tau, the main components of plaques and tangles respectively, decades ago has provided great hope for disease-modifying drugs, but yet there are no curative treatments. Currently, available drugs for the AD are only symptomatic ones, which are effective for a limited time without altering the course of disease progress. The extracellular plaque deposits of the β-amyloid peptide (Aβ) and the neurofibrillary tangles of the microtubule binding protein tau inside neuron are widely considered as two major pathological hallmarks required for a diagnosis of AD. So far, clinical trials to test promising new treatments aimed at amyloid β (Aβ) and tau protein using monoclonal antibodies and small molecules have yielded disappointing results (clinical trials, Merck & Lily, 2016, 2017 and LM™ by TauRx, 2016). For example, a number of large-scale clinical trials have been performed to evaluate new treatments aimed at soluble Aβ as well as insoluble aggregates, but the results have been disappointing so far.^{1,2} Similarly, tau-directed drug development approach has yet to yield promising therapeutics.^{3,4} As a result, alternative processes not directly connected to Aβ or tau pathways have been of substantial interest to researchers. For example, interest has been growing in other targets such as components of innate inflammatory pathways.

In response to cellular stress or pro-inflammatory cytokines such as TNF-α or interferon (INF)-γ, cells upregulate variant forms of proteasome catalytic subunits, known as immuno-subunits. As a result, cells undergo dynamic changes in proteasome assembly to form the immunoproteasome (iP), which harbors immuno-subunits LMP7, LMP2 and Mecl-1 instead of constitutive counterparts X, Y and Z, respectively. It has been reported that overall activity of the iP is considerably enhanced compared to the constitutive proteasome (cP)⁵ and that shaping the antigenic repertoire of MHC class I is a major function of the iP.⁶ The iP was also shown to be vital for the degradation of misfolded and oxidant-damaged proteins to prevent disease progress.^{5,7} A selective inhibitor of LMP7 is shown to block cytokine production and attenuate progression of experimental arthritis in mouse models,⁸ implicating an important role of LMP7 during inflammation. Although the cellular functions of other immune-subunits, LMP2 and Mecl-1, are not actively investigated so far, recent studies report that LMP2 has no role in pro-inflammatory cytokine production or NF-kB activation in cancer cells and human peripheral blood mononuclear cells.^{9,10}

The proteasome appears to play a role in the pathogenesis of neurodegenerative diseases such as AD. Several previous studies reported significant alterations in proteasome activity and accumulation of ubiquitinated protein deposits in the brains and cerebrospinal fluid of AD patients and in rodent models of disease.¹¹⁻¹⁴ In in vitro studies, Aβ peptides have been shown to reduce the activity of 20S proteasomes, while increasing activity of the 20S proteasomes capped with the 19S and/or 11S regulators. Accumulation of insoluble tau is also shown to be associated with a decrease in the catalytic activity of brain 26S proteasomes and higher levels of ubiquitinated proteins.¹⁵ On the other hand, other researchers reported rather disparate observations that elevated IP expression and activity is positively correlate with increasing severity of tau pathology and microglial activation in AD patients as well as in a mouse model of brain injury.¹⁶⁻¹⁹ Similarly, elevated expression of iP was spotted in microglia and astrocytes surrounding Aβ plaques isolated from AD animals.²⁰ In a mouse model of Aβ deposition, LMP7 knockout resulted in alterations in microglial cytokine production profile and improved cognitive deficits,²¹ indicating a role of LMP7 subunit in Aβ-induced neuroinflammation.

While the LMP7, which is responsible for CT-L activity, has drawn considerable interest as a potential therapeutic target for inflammatory diseases, LMP2, which also cleaves after hydrophobic amino acid residues, is not considered a therapeutic target. It has been suggested that the contribution of LMP2 to overall CT-L activity of iP is relatively small compared to that of LMP7.²² This was further supported by recent studies showing ineffectiveness of LMP2-selective inhibitors on inhibition of cytokine production.³ For this reason, LMP2 has not been actively pursued as a therapeutic target in autoimmune diseases. Regarding the role of LMP2 in neurodegenerative diseases, there have so far been very few studies conducted, reporting moderate up-regulation of LMP2 expression in AD patients.^{18,23}

There is still much work to be done to provide proteasome inhibitors which overcome intrinsic and acquired drug resistance, which have clinical utility outside of multiple myeloma, and which have improved pharmacokinetic properties.

Accordingly, the subject matter of the present disclosure relates to the development of unique proteasome inhibitors that have improved pharmacokinetic properties and broader and/or unique treatment applications as compared to compounds known in the art.

### SUMMARY

The presently-disclosed subject matter further includes a pharmaceutical composition, which includes at least one compound according to formula (I), (II), (III), or formula (IV), and a pharmaceutically-acceptable carrier.

The presently-disclosed subject matter also includes a method of inhibiting a proteasome in a cell, which involves administering or contacting the compound of formula (I), (II), (III), or formula (IV), to the cell. In some embodiments, the compound of formula (IV) is administered as a general proteasome inhibitor. In some embodiments, a compound according to formula (I), (II), or (III), or a combination thereof, is administered as an immunoproteasome subunit LMP2 inhibitor.

In some embodiments, the compound is a proteasome subunit LMP2 inhibitor or a general proteasome inhibitor. In some embodiments, the unique proteasome inhibitor is a compound of formula (I), a pharmaceutically acceptable salt, solvate, or physiologically functional derivative thereof:

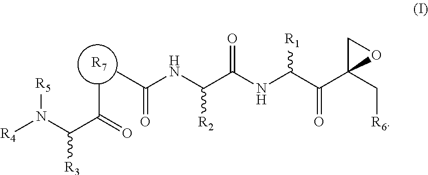

In some embodiments, the unique proteasome inhibitor is a compound of formula (II), a pharmaceutically acceptable salt, solvate, or physiologically functional derivative thereof:

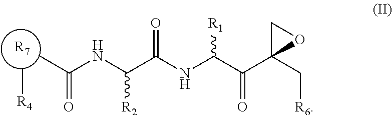

In some embodiments, the unique proteasome inhibitor is a compound of formula (III), a pharmaceutically acceptable salt, solvate, or physiologically functional derivative thereof:

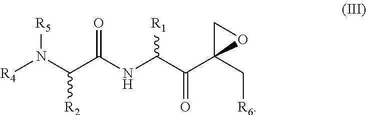

In some embodiments, the unique proteasome inhibitor is a compound of formula (IV), a pharmaceutically acceptable salt, solvate, or physiologically functional derivative thereof:

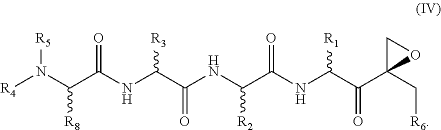

The presently-disclosed subject matter also includes a method of treating a disease in a subject, which includes administering an effective amount of a pharmaceutical composition containing the compound of formula (I), formula (II), formula (III) or formula (IV) to the subject. The disease can be, for example, a neurodegenerative disease, an autoimmune disease, or cancer. In some embodiments, the disease is Alzheimer's Disease (AD), age-related macular degeneration (AMD) or Multiple Myeloma (MM). In some embodiments, the administering results in improved memory function. In some embodiments, the disease is relapsed/refractory MM and/or is resistant to carflizomib and/or bortezomib.

In some embodiments, the administering is to a cell, in some instances the cell is a cancer cell or a retinal pigment epithelial cell. In other instances, the administering is to a subject.

### BRIEF DESCRIPTION OF THE DRAWINGS

The unique features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIG. 1 shows the initial screening for immunoproteasome catalytic subunit LMP2-Specific Inhibitors.

FIG. 2 charts the results of the initial screen of inhibitors targeting the immunoproteasome catalytic subunit LMP2. The initial Screen of Inhibitors: Testing In Triplicate At 0.3 and 0.1 uM against 20S cP and 20S iP with 100 uM Ac-nLPnLD-AMC and 100 uM Ac-PAL-AMC as substrates, respectively.

FIG. 3A illustrates structural features of potent LMP2 Inhibitors, FIG. 3B provides IC₅₀ measurements in μM for β1i, β1, β5 and β5i and FIG. 3C shows LMP2 activity.

FIG. 4 shows strategy for optimization of proteasome inhibitors with P1′ residue.

FIG. 5 documents efficacy of treatments against multiple myeloma (MM) samples resistant to FDA approved proteasome inhibitors carfilzomib and bortezomib with the associated structures of the inhibitors.

FIG. 6 includes images of retinal pigment epithelium (RPE) of Alzheimer's Disease (AD) mouse model (APP mutant) control or treated with YU102 and RPE were incubated with β-catenin or E-cadherin, subsequently incubated with Alexa 48, 555-conjugated secondary antibody and nuclei stained by Torpo-3. Samples observed with Olympus, FV-1000 spectral confocal microscope.

FIG. 7 charts the effect of YU102 on memory impairment of the APPsw mouse model.

FIG. 8 charts the effect of YU102 on LPS-induced memory impairment.

FIG. 9A is the ELISA-based quantification of Aβ; and FIG. 9B provides the thioflavin T staining of Aβ, showing that the efficacy of YU102 in AD models is independent of Aβ deposition.

FIG. 10 includes images showing YU102 displays no neuroprotective effects in the APPsw mouse model.

FIG. 11A includes images showing YU102 inhibits activation of astrocytes; and FIG. 11B includes images showing YU102 inhibits activation of microglia via suppression of neuroinflammation responses.

FIG. 12A-12D charts how the inhibition of LMP2 ameliorates cognitive deficits in the APPsw mice (Both compound #16 and YU102 are selective LMP2 inhibitor).

FIG. 13 charts how LMP2 inhibitors do not inhibit tau aggregation. Low tau-BiFC intensity observed at high concentration of LMP2 inhibitors (30-100 μM) were due to cell death. YU102 epimer is an inactive stereoisomer of YU102.

FIG. 14 charts LMP2 inhibition resulting in impairments in LPS-induced production of interleukin-6 (IL-6) in BV-2 cells.

FIG. 15 provide images of RPE tissues showing LMP2 inhibitors block RPE degeneration. Retinal pigment epithelium (RPE) of AD mice (APPsw) was isolated and incubated with β-catenin (1:100 diluted) overnight at 4° C. After incubation with primary antibody, the RPE tissues were further incubated with Alexa 555-conjugated secondary antibody (Invitrogen; A21422; 1:1000 diluted) at room temperature for 2 hours. Sample was observed by using a confocal microscope (Carl Zeiss, LSM 800).

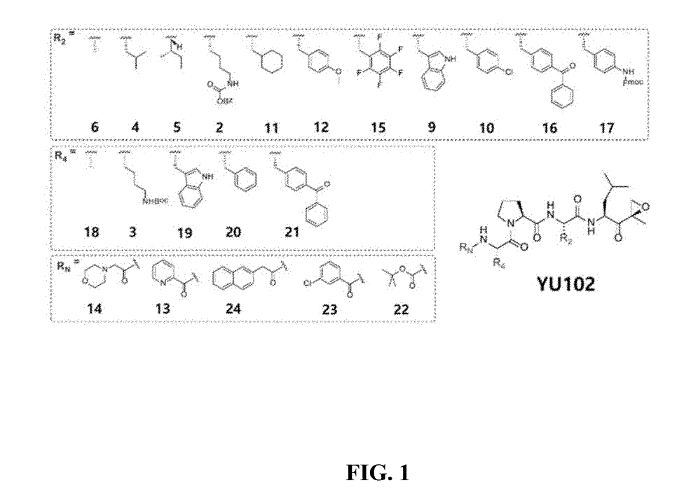

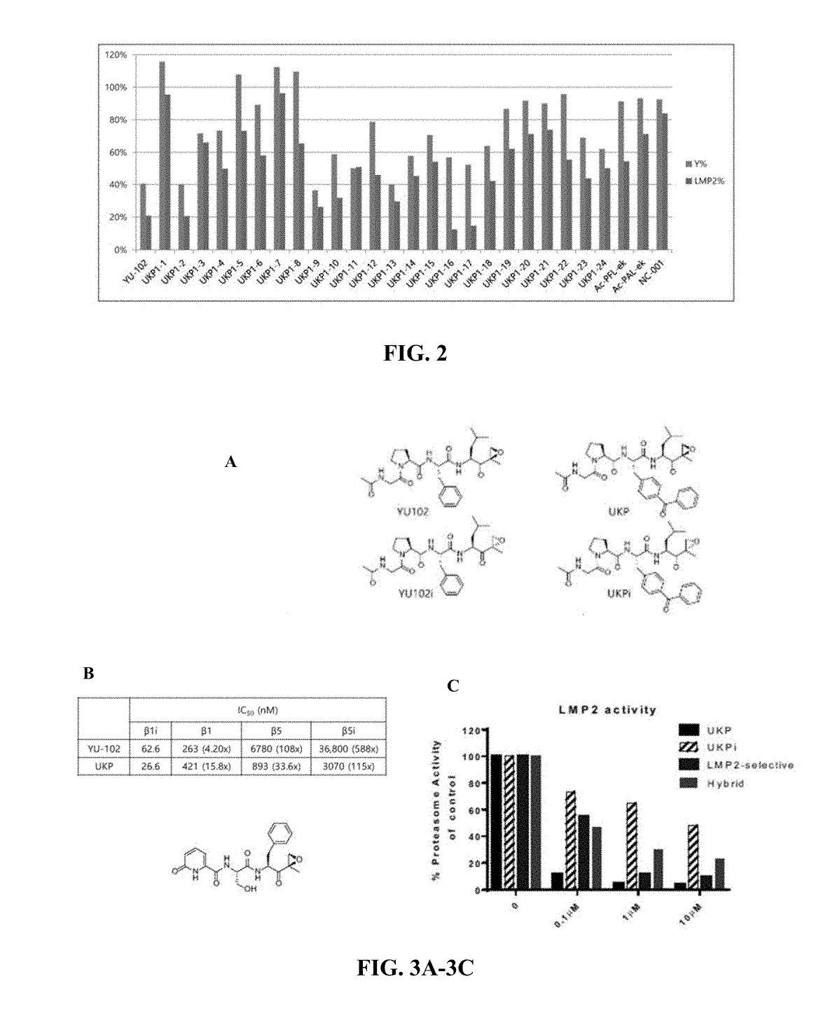

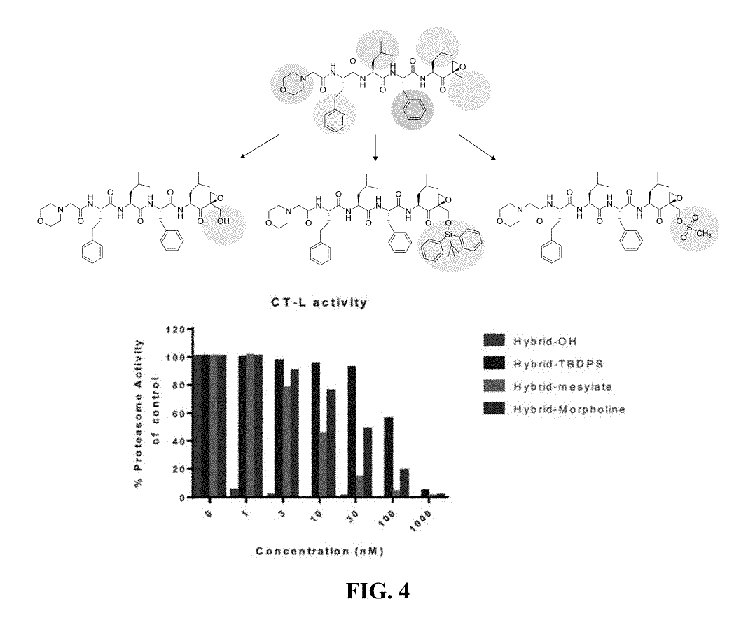

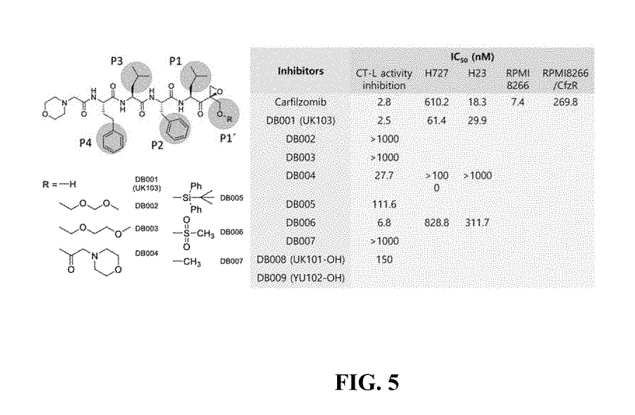

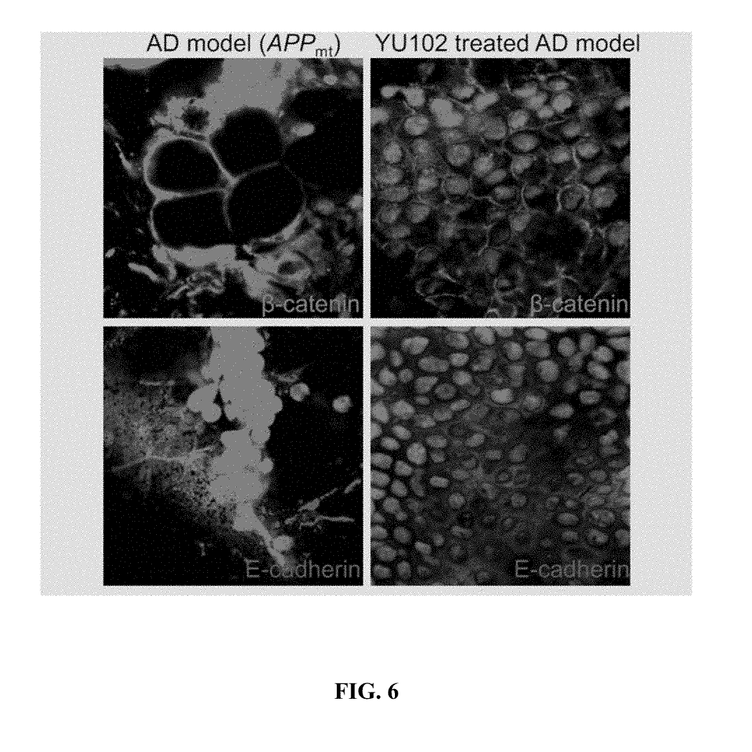

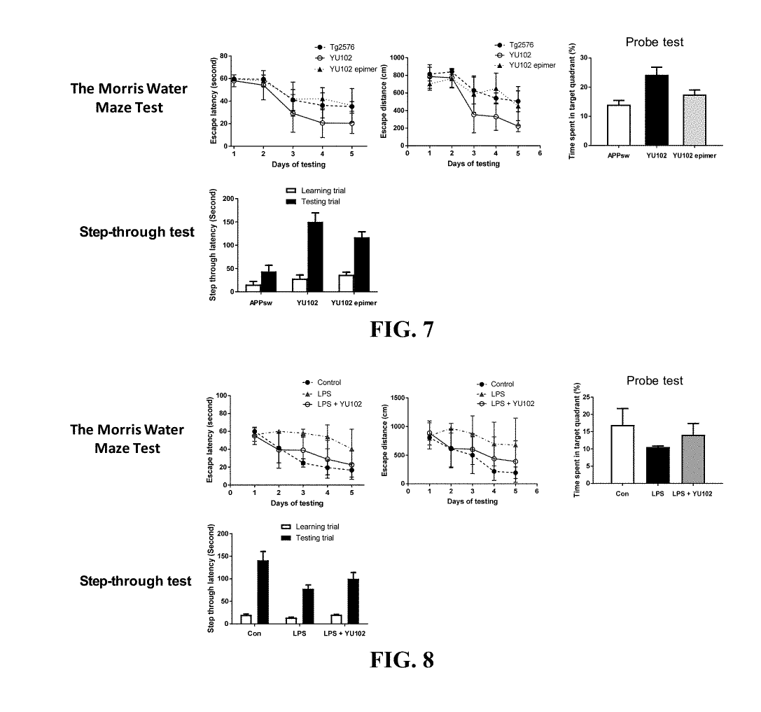

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by those of ordinary skill in the art, certain definitions are set forth to facilitate explanation of the presently-disclosed subject matter. Unless defined otherwise, all technical and scientific terms used herein have the same meaning as commonly understood by one of ordinary skill in the art to which the presently-disclosed subject matter belongs.

Although any methods, devices, and materials similar or equivalent to those described herein can be used in the practice or testing of the presently-disclosed subject matter, representative methods, devices, and materials are now described.

The present disclosure includes inhibitors of the 20S proteasome, specifically to a series of proteasome-inhibiting peptide epoxyketones. The current compounds, unlike previous FDA-approved proteasome-inhibiting peptide epoxyketones, include a functional group appended to the epoxy group on the peptide epoxyketones, denoted as the P1′ position.

As disclosed herein, patient multiple myeloma (MM) samples that are resistant to approved inhibitors, such as bortezomib and carfilzomib, have responded to inhibitors of the currently-disclosed class of peptide epoxyketones. Additionally, peptide epoxyketones, particularly those targeting immunoproteasome catalytic subunits, display efficacy against in vivo Alzheimer's Disease (AD) models.

The presently disclosed subject matter includes a unique proteasome inhibitor, including a compound of formula (I), (II), (III), or (IV), or a pharmaceutically acceptable salt, solvate, or physiologically functional derivative thereof.

In some embodiments, the compound is a proteasome subunit LMP2 inhibitor or a general proteasome inhibitor. In some embodiments, the unique proteasome inhibitor is a compound of formula (I), a pharmaceutically acceptable salt, solvate, or physiologically functional derivative thereof:

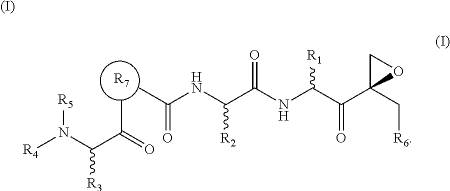

In some embodiments, the unique proteasome inhibitor is a compound of formula (II), a pharmaceutically acceptable salt, solvate, or physiologically functional derivative thereof:

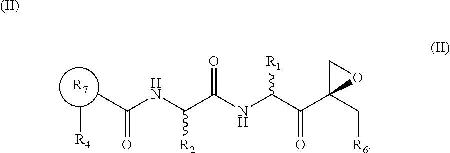

In some embodiments, the unique proteasome inhibitor is a compound of formula (III), a pharmaceutically acceptable salt, solvate, or physiologically functional derivative thereof:

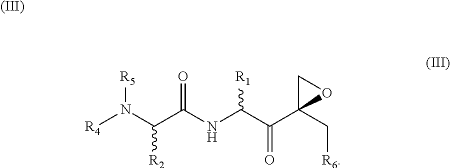

In some embodiments, the unique proteasome inhibitor is a compound of formula (IV), a pharmaceutically acceptable salt, solvate, or physiologically functional derivative thereof:

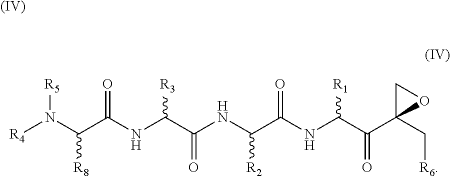

In some embodiments of a compound of formulae (I), (II), (III) or (IV), R₁, R₂, R₃ and R₈ are independently selected from

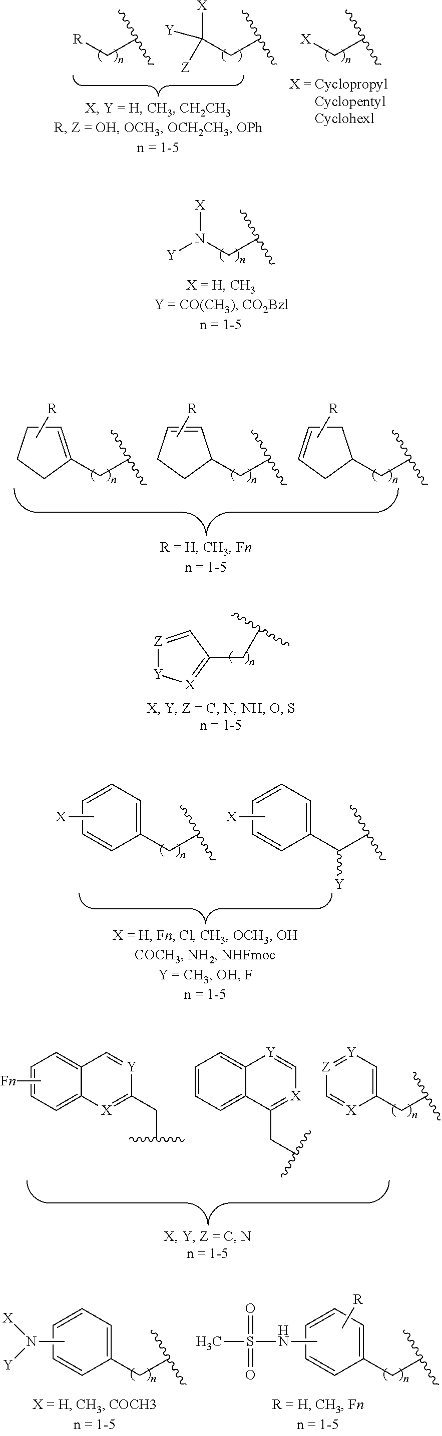

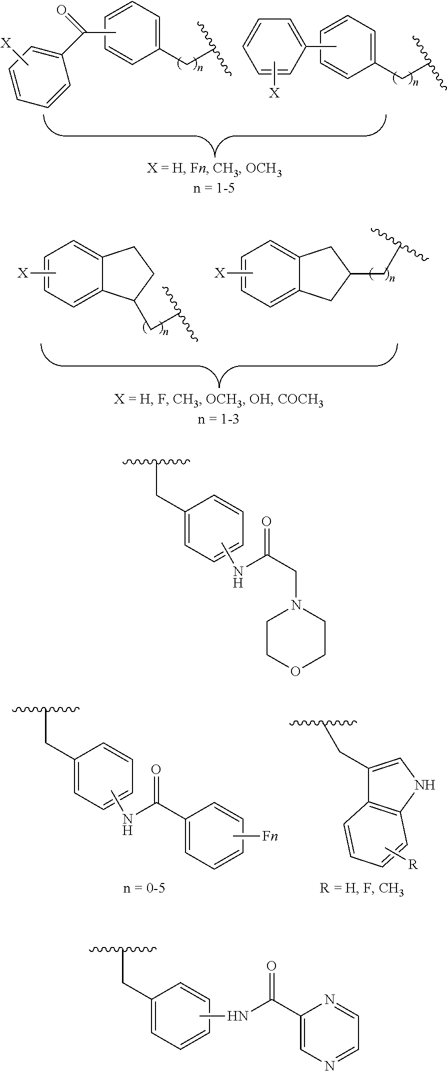

In some embodiments of a compound of formulae (I), (II), (III) or (IV), R₄ is selected from

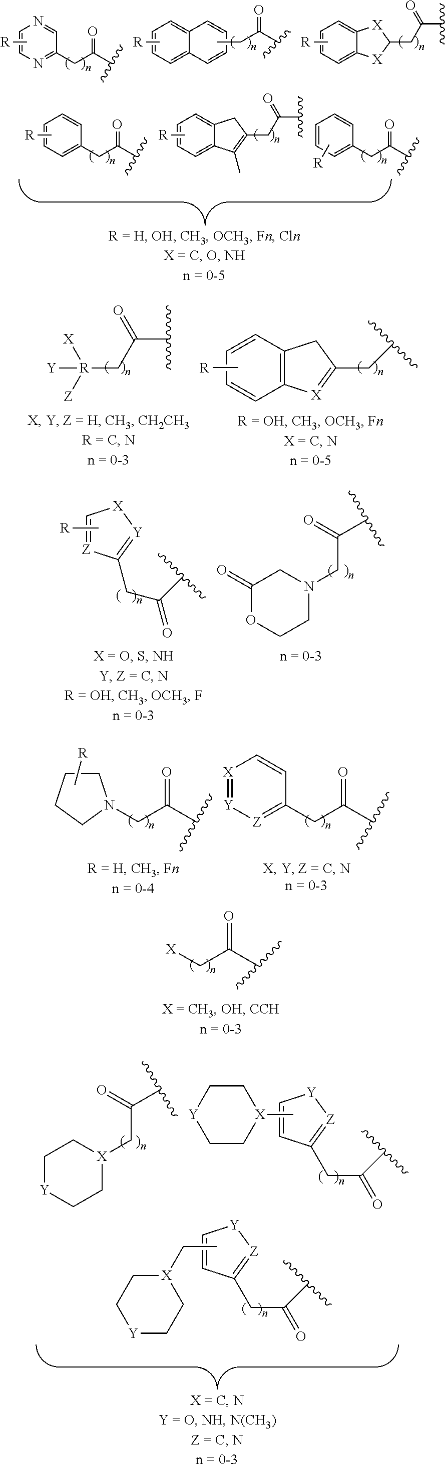

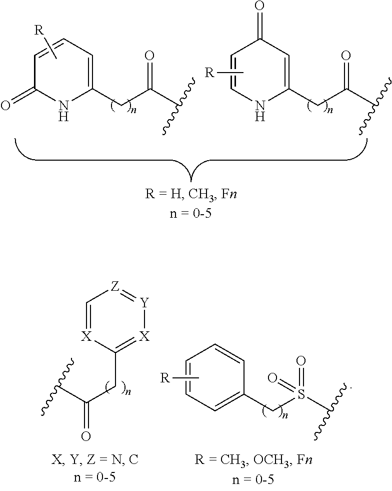

In some embodiments of a compound of formulae (I), (III) or (IV), R₅ is selected from H and CH₃.

In some embodiments of a compound of formulae (I), (II), (III) or (IV), R₆ is selected from

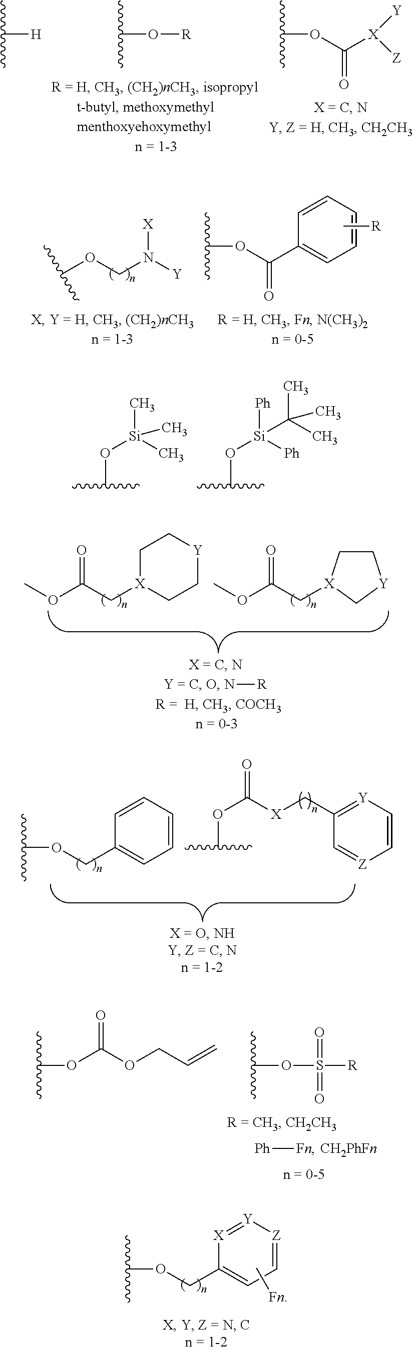

In some embodiments of a compound of formulae (I) or (II), R₇ is selected from

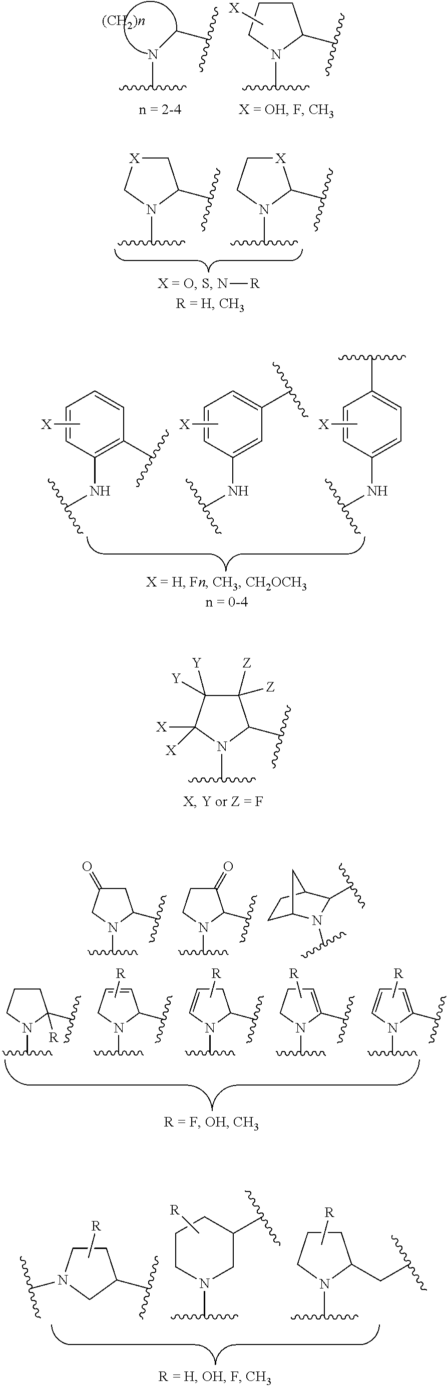

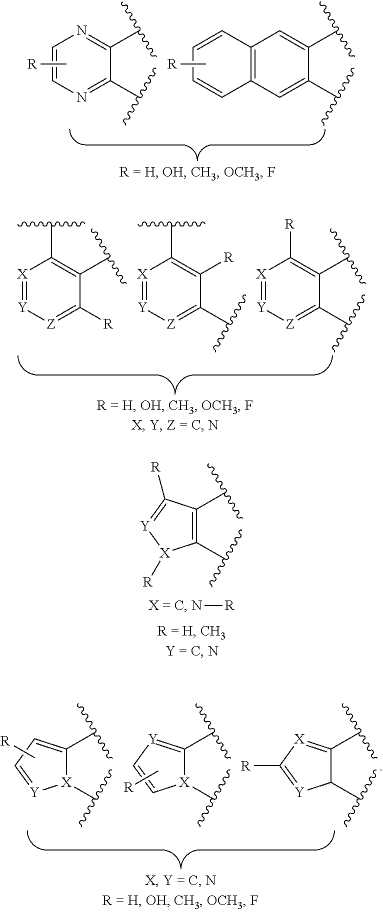

In a preferred embodiment of the compound of formula (III) or (IV), R₅ are H.

The presently-disclosed subject matter further includes a pharmaceutical composition, which includes at least one compound according to formula (I), (II), (III), or formula (IV), and a pharmaceutically-acceptable carrier.

The presently-disclosed subject matter also includes a method of inhibiting a proteasome in a cell, which involves administering or contacting the compound of formula (I), (II), (III), or formula (IV), to the cell. In some embodiments, the compound of formula (IV) is administered as a general proteasome inhibitor. In some embodiments, a compound according to formula (I), (II), or (III), or a combination thereof, is administered as an immunoproteasome LMP2 inhibitor. The administering or contacting the compound to the cell can be, for example, a cancer cell. In other embodiments, the administration of contacting the compound to the cell can be to a Retinal Pigment Epithelium (RPE) cell.

The presently-disclosed subject matter also includes a method of treating a disease in a subject, which includes administering an effective amount of a pharmaceutical composition containing the compound of formula (I), formula (II), formula (III) or formula (IV) to the subject. The disease can be, for example, a neurodegenerative disease, an autoimmune disease, or cancer. In some embodiments, the disease is Alzheimer's Disease (AD), age-related macular degeneration (AMD) or Multiple Myeloma (MM). In some embodiments, the disease is relapsed/refractory MM.

Unless otherwise indicated, the term “administering” is inclusive of all means known to those of ordinary skill in the art for providing a preparation to a subject, including administration by inhalation, nasal administration, topical administration, intravaginal administration, ophthalmic administration, intraaural administration, intracerebral administration, intravitreous administration, intracameral administration, posterior sub-Tenon administration, posterior juxtascleral administration, subretinal administration, suprachoroidal administration, cell-based administration or production, rectal administration, and parenteral administration, including injectable such as intravenous administration, intra-arterial administration, intramuscular administration, and/or subcutaneous administration. Administration can be continuous or intermittent. A preparation can be administered therapeutically; that is, administered to treat an existing condition of interest. A preparation can be administered prophylactically; that is, administered for prevention of a condition of interest.

In some embodiments a subject will be administered an effective amount of at least one compound provided in the present disclosure. In this respect, the term “effective amount” refers to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

Additionally, the terms “subject” or “subject in need thereof” refer to a target of administration, which optionally displays symptoms related to a particular disease, pathological condition, disorder, or the like. The subject of the herein disclosed methods can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. A patient refers to a subject afflicted with a disease or disorder. The term “subject” includes human and veterinary subjects.

The term “physiologically functional derivative” means any pharmaceutically acceptable derivative of a compound of the present disclosure. For example, an amide or ester of a compound of formula (I), which upon administration to a subject, particularly a mammal, is capable of providing, either directly or indirectly, a compound of the present disclosure of an active metabolite thereof.

As will be recognized by one of ordinary skill in the art, the terms “suppression,” “suppressing,” “suppressor,” “inhibition,” “inhibiting” or “inhibitor” do not refer to a complete elimination of a value in all cases. Rather, the skilled artisan will understand that the term “suppressing” or “inhibiting” refers to a reduction or decrease in a measured value, qualitatively or quantitatively. Such reduction or decrease can be determined relative to a control. In some embodiments, the reduction or decrease relative to a control can be about a 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% decrease.

In some embodiments the subject in need thereof will be suffering or will have been diagnosed with one or more neoplastic or hyperproliferative diseases, disorders, pathologies, or conditions. Examples of such diseases, conditions, and the like include, but are not limited to, neoplasms (cancers or tumors) located in the colon, abdomen, bone, breast, digestive system, esophagus, liver, pancreas, peritoneum, endocrine glands (adrenal, parathyroid, pituitary, testicles, ovaries, cervix, thymus, thyroid), eye, head and neck, nervous (central and peripheral), lymphatic system, pelvis, skin, soft tissue, spleen, thoracic areas, bladder, and urogenital system. Other cancers include follicular lymphomas, carcinomas with p53 mutations, and hormone-dependent tumors, including, but not limited to colon cancer, cardiac tumors, pancreatic cancer, melanoma, retinoblastoma, glioblastoma, lung cancer, intestinal cancer, testicular cancer, stomach cancer, neuroblastoma, myxoma, myoma, lymphoma, endothelioma, osteoblastoma, osteoclastoma, osteosarcoma, chondrosarcoma, adenoma, breast cancer, prostate cancer, Kaposi's sarcoma and ovarian cancer, or metastases thereof.

A subject may also be in need thereof because they have acquired diseases or conditions such as autoimmune diseases such as, but not limited to, Addison's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune pancreatitis, Autoimmune retinopathy, Autoimmune urticaria, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss, Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multiple sclerosis, Myasthenia gravis, Myositis, Narcolepsy, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonnage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatic, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, and Wegener's granulomatosis (or Granulomatosis with Polyangiitis (GPA)).

A subject may also be in need thereof because they have neurodegenerative conditions such as Alzheimer's Disease, amyotrophic lateral sclerosis, Parkinson's, and Huntington's. A subject may also be in need thereof because of conditions such as age-related macular degeneration.

As used herein, the terms “treatment” or “treating” relate to any treatment of a condition of interest, including but not limited to prophylactic treatment and therapeutic treatment. As such, the terms treatment or treating include, but are not limited to: preventing a condition of interest or the development of a condition of interest; inhibiting the progression of a condition of interest; arresting or preventing the development of a condition of interest; reducing the severity of a condition of interest; ameliorating or relieving symptoms associated with a condition of interest; and causing a regression of the condition of interest or one or more of the symptoms associated with the condition of interest.

Moreover, the subject matter of the present disclosure relates to the development of unique proteasome inhibitors that utilize a peptide epoxyketone scaffold.

In some embodiments, the unique proteasome inhibitors of the present disclosure comprise a substituted peptide epoxyketone scaffold, which provides for relatively potent inhibition of proteasome. In some embodiments, the compound, a pharmaceutically acceptable salt, solvate, or physiologically functional derivative thereof can be any analog from Table 2.

TABLE 1
Proteasome inhibitory activity of LMP2 inhibitors.

Subunit-Specific Cell lysate IC₅₀ (μM)

| | Chymotrypsin-like | |
|---|---|---|
| Analogue | activity (β5/β5i) | LMP2 (β1i) |
| YU-102 | >10 | 0.10 |
| 1 | >10 | 1.20 |
| 2 | >10 | 1.0 |
| 3 | >10 | 1.0 |
| 4 | >10 | 0.65 |
| 5 | >10 | >10 |
| 6 | 7 | 2.20 |
| 7 | >10 | 0.03 |
| 8 | 5 | 0.02 |
| 9 | >10 | 0.13 |
| 10 | >10 | 0.03 |
| 11 | >10 | 0.06 |
| 12 | >10 | 0.07 |
| 13 | >10 | 0.06 |
| 14 | 1 | 0.12 |
| 15 | >10 | 0.10 |
| 16 | >10 | 0.08 |
| 17 | >10 | 0.14 |
| YU-102 | >10 | 1.16 |
| 18 | 5 | 0.07 |
| 19 | >10 | 0.18 |
| 20 | 10 | 0.10 |
| 21 | >10 | 0.13 |
| 22 | 5 | 0.09 |
| 23 | 10 | 0.06 |
| 24 | 10 | 0.13 |
| 25 | >10 | 0.13 |
| 26 | 10 | 0.09 |
| 27 | >10 | 0.11 |
| 28 | >10 | 0.03 |
| 29 | >10 | 0.09 |
| 30 | 10 | 0.12 |

In some embodiments, the subject matter of the present disclosure is directed to proteasome inhibitors that have improved pharmacokinetic properties and broader treatment applications than those previously known in the art. In some embodiments, the peptide proteasome inhibitors of the present disclosure demonstrate activity against multiple myeloma with acquired resistance to bortezomib and/or carfilzomib, neurodegenerative disease such as AD, and AMD.

Certain compounds of formulae disclosed herein may exist in stereoisomeric forms (e.g., they may contain one or more asymmetric carbon atoms, or they may exhibit cis-trans isomerism), and, the individual stereoisomers and mixtures of these are included within the scope of the present disclosure.

The unique proteasome inhibitors of the present disclosure may be used for the treatment of a disease or condition, such as cancer. In some embodiments, there is provided a pharmaceutical composition for use in the treatment (including prophylaxis) of one or more conditions or indications set forth herein, which comprises a compound of formula ((I), (II), (III), or (IV), a pharmaceutically acceptable salt, solvate, or physiologically functional derivative thereof, and a pharmaceutically acceptable carrier or excipient.

Processes for preparing a pharmaceutically acceptable salt, solvate and/or a physiologically functional derivative of the compound(s) of formula (I), (II), (III), or (IV) are disclosed herein.

The presently-disclosed subject matter further includes pharmaceutical compositions of the compounds as disclosed herein, and further includes a pharmaceutically-acceptable carrier. In this regard, the term “pharmaceutically acceptable carrier” refers to sterile aqueous or non-aqueous solutions, dispersions, suspensions or emulsions, as well as sterile powders for reconstitution into sterile injectable solutions or dispersions just prior to use. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Injectable formulations are also prepared by entrapping the drug in liposomes or microemulsions, which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose.

Suitable formulations include aqueous and non-aqueous sterile injection solutions that can contain antioxidants, buffers, bacteriostats, bactericidal antibiotics and solutes that render the formulation isotonic with the bodily fluids of the intended recipient; and aqueous and non-aqueous sterile suspensions, which can include suspending agents and thickening agents.

The compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

The formulations can be presented in unit-dose or multi-dose containers, for example sealed ampoules and vials, and can be stored in a frozen or freeze-dried condition requiring only the addition of sterile liquid carrier immediately prior to use.

For oral administration, the compositions can take the form of, for example, tablets or capsules prepared by a conventional technique with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycollate); or wetting agents (e.g., sodium lauryl sulphate). The tablets can be coated by methods known in the art.

Liquid preparations for oral administration can take the form of, for example, solutions, syrups or suspensions, or they can be presented as a dry product for constitution with water or other suitable vehicle before use. Such liquid preparations can be prepared by conventional techniques with pharmaceutically acceptable additives such as suspending agents (e.g., sorbitol syrup, cellulose derivatives or hydrogenated edible fats); emulsifying agents (e.g. lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate. Preparations for oral administration can be suitably formulated to give controlled release of the active compound. For buccal administration the compositions can take the form of tablets or lozenges formulated in conventional manner.

The compounds can also be formulated as a preparation for implantation or injection. Thus, for example, the compounds can be formulated with suitable polymeric or hydrophobic materials (e.g., as an emulsion in an acceptable oil) or ion exchange resins, or as sparingly soluble derivatives (e.g., as a sparingly soluble salt).

The compounds can also be formulated in rectal compositions (e.g., suppositories or retention enemas containing conventional suppository bases such as cocoa butter or other glycerides), creams or lotions, or transdermal patches.

The presently-disclosed subject matter further includes a kit that can include a compound or pharmaceutical composition as described herein, packaged together with a device useful for administration of the compound or composition. As will be recognized by those or ordinary skill in the art, the appropriate administration-aiding device will depend on the formulation of the compound or composition that is selected and/or the desired administration site. For example, if the formulation of the compound or composition is appropriate for injection in a subject, the device could be a syringe. For another example, if the desired administration site is cell culture media, the device could be a sterile pipette.

Still further, the presently-disclosed subject matter includes a method for treating cancer. In some embodiments the method comprises administering a compound, including one of the compounds described herein, to a subject in need thereof. In some embodied methods a plurality of compounds according the present disclosure are administered simultaneously or in a predetermined sequence.

There are also provided processes for the preparation of a non-peptide proteasome inhibitor according to the present disclosure. For example, in some embodiments, the present disclosure provides processes for the preparation of a compound of formula (I), (II), (III), or (IV), a pharmaceutically acceptable salt, solvate, or physiologically functional derivative thereof.

Additionally, the present disclosure provides uses of a compound of formula (I), (II), (III), or (IV) a salt, a solvate, or physiological derivative thereof in the preparation or manufacture of a drug and/or medicine, especially a medicine for the treatment of cancer, neurodegenerative disease, or autoimmune disease in a mammal.

In some embodiments, the present disclosure provides methods for treating a subject with neurodegenerative disease, autoimmune disease, or cancer by administering to the subject an effective amount of at least one proteasome inhibitor.

In some embodiments, the presently-disclosed subject matter provides a proteasome inhibitor comprising at least one peptide epoxyketone. In some embodiments, the proteasome inhibitor is an epoxyketone that generally inhibits the proteasome or is selective for LMP2.

In some embodiments, the presently-disclosed subject matter provides a method of inhibiting a proteasome in a cell, which involves administering an effective amount of a compound of formula (I), (II), (III), or (IV) to the cell.

In certain embodiments, the present disclosure provides a method of treating a disease, wherein the method comprises administering to a subject at least one proteasome inhibitor, wherein the proteasome inhibitor comprises at least one peptide epoxyketone.

In certain embodiments, the present disclosure provides a method of treating a disease, wherein the method comprises administering to a subject an effective amount of a pharmaceutical composition containing at least one compound according to formula (I), (II), (III), or (IV).

In some embodiments, the present disclosure is directed to a pharmaceutical composition comprising at least one proteasome inhibitor, wherein the proteasome inhibitor includes at least one peptide epoxyketone.

In some embodiments, the present disclosure is directed to a pharmaceutical composition comprising at least one compound according to formula (I), (II), (III), or (IV).

In some embodiments, the present disclosure teaches a method of synthesizing a proteasome inhibitor comprising at least one peptide epoxyketone.

Further, the present disclosure provides, in certain embodiments, a method of treating a disease in a subject comprising the administration of an effective amount of a pharmaceutical composition containing a protease inhibitor, a pharmaceutically acceptable salt, solvate or physiologically functional derivative thereof and pharmaceutically acceptable excipient to the subject.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

The presently-disclosed subject matter is further illustrated by the following specific but non-limiting examples. The following examples may include compilations of data that are representative of data gathered at various times during the course of development and experimentation related to the present invention.

### EXAMPLES

A new class of peptide epoxyketones that have shown impressive efficacies against relapsed/refractory multiple myeloma and neurodegenerative diseases such as Alzheimer's disease are reported herein. Unlike peptide epoxyketones previously reported and patented, this new class of peptide epoxyketones contain P1′ groups, a unique feature that is lacking in FDA-approved proteasome inhibitor drugs. Some of this class of inhibitors showed efficacy against patient MM samples that were resistant to FDA-approved proteasome inhibitors bortezomib and carfilzomib. In addition, peptide epoxyketones targeting immunoproteasome catalytic subunits showed impressive efficacy against in vivo AD models, such as LPS and swAPP transgenic mouse models.

### Example 1: Development of Next-Generation Proteasome Inhibitors (PIs)

Next-Generation PIs that are Highly Effective Against Animal Models of Neurodegenerative Diseases and Relapsed/Refractory MM.

Next-generation of peptide epoxyketones developed by us were highly effective against animal models of neurodegenerative diseases. Unlike FDA-approved carfilzomib that targets multiple proteasome catalytic subunits, these PIs were selective towards particular catalytic subunits and significantly less cytotoxic, compared to cafilzomib.

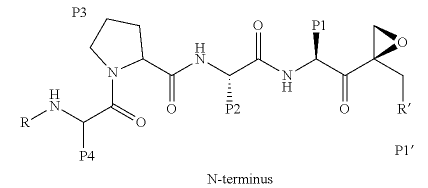

Representative Structure of New Generation Proteasome Inhibitors.

Optimization of Lead PIs for Improving PK Properties.

To improve the PK profiles and the potency of our lead PIs, we prepared a small library of PIs based on the backbone of our lead PIs, specifically focusing on P4 and P2 positions. In addition, the P3 position was further optimized, using a variety of ring structures, such as thiazole or piperidine. Derivatization at these positions yielded PIs with an improved potency and aqueous solubility.

PIs with an Improved Metabolic Stability.

Currently, the poor in vivo stability associated with peptide epoxyketones has been attributed to the high susceptibility to peptidases (hydrolyzing peptide bonds within PIs) and epoxide hydrolases (hydrolyzing the epoxide pharmacophore). To improve metabolic stability of lead PIs, we first introduced a P1′ moiety into lead PIs, anticipating that the P1′ residue would interfere with the access of epoxide pharmacophore into the narrow, hydrophobic active site of epoxide hydrolases, inhibiting the hydrolysis of epoxide and prolonging the activity of PIs. Our initial data showed that P1′ derivatives of lead PIs have considerably improved metabolic stability compared to PIs without P1′ residue.

Synthesis of Epoxyketones for Formula I-IV Compounds

Reaction of protected aminoacid methyl ester with tert-butyllithium and dimethyl methylphosphonate followed by combination of Wittig-Horner and Baylis-Hillman-type reaction furnished hydroxymethyl enone 1 in good yield. Simple addition of R₆ and subsequent epoxidation yielded epoxides 2. Alternatively, compound 2 was prepared from amino acids.

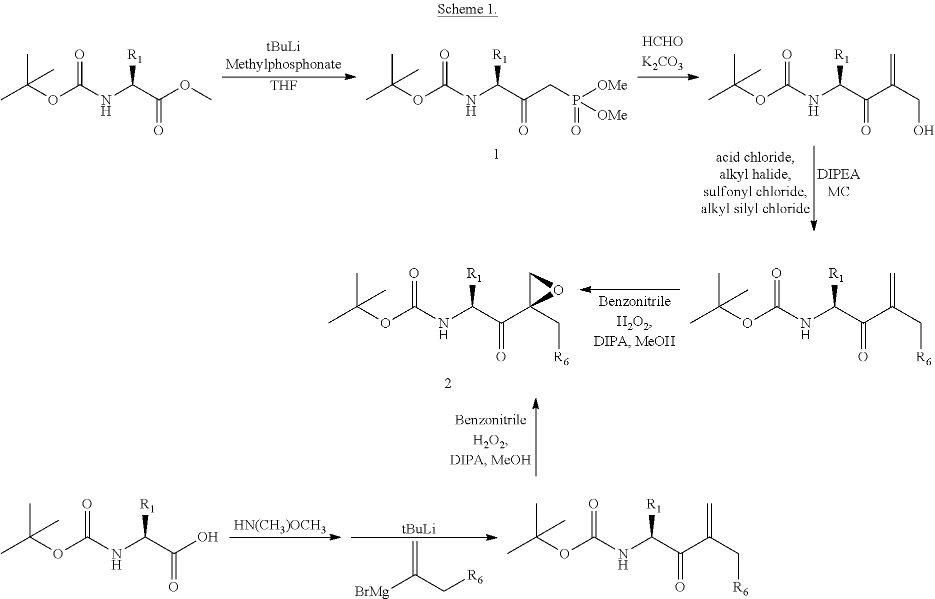

Synthesis of Formula I Compounds

Dipeptide 3 was prepared by coupling two amino acids using coupling agents (HOBt and HBTU) in the presence of organic base DIEA. Deprotection of Boc followed by another peptide coupling reaction gave tripeptide 4. Removal of Boc from tripeptide 4 in the presence of TFA and methylene chloride and subsequent amide coupling or alkylation or acylation furnished peptide backbone 5.

Finally, acid-deprotected compounds 5 were coupled with Boc-deprotected epoxides (2) to generate the final compounds.

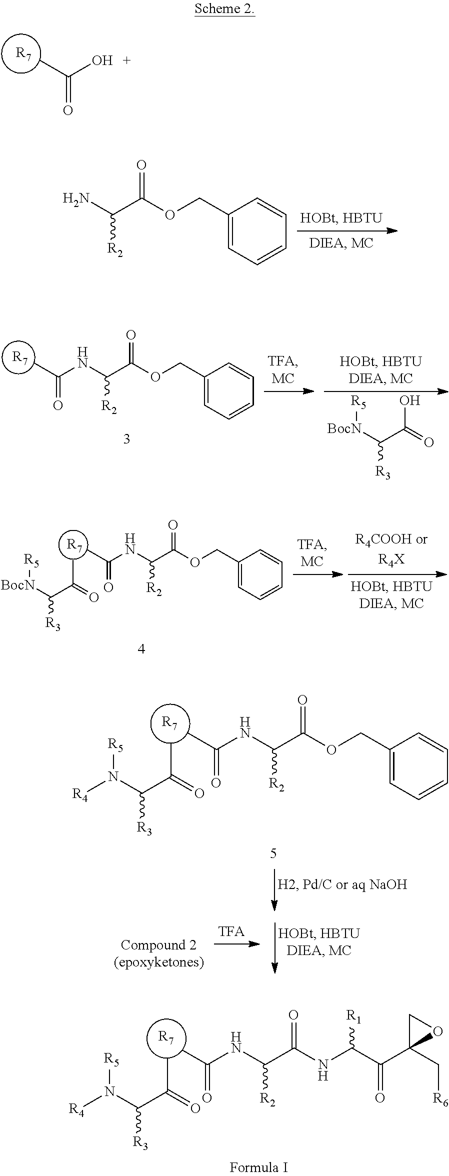

Synthesis of Formula II Compounds

Dipeptide 6 was prepared by coupling two amino acids using coupling agents (HOBt and HBTU) in the presence of organic base DIEA. Deprotection of Boc from dipeptide 6 in the presence of TFA and methylene chloride followed by amide coupling or alkylation or acylation furnished peptide backbone 7.

Finally, acid deprotected compounds 7 were coupled with Boc-deprotected epoxides (2) to generate the final compounds.

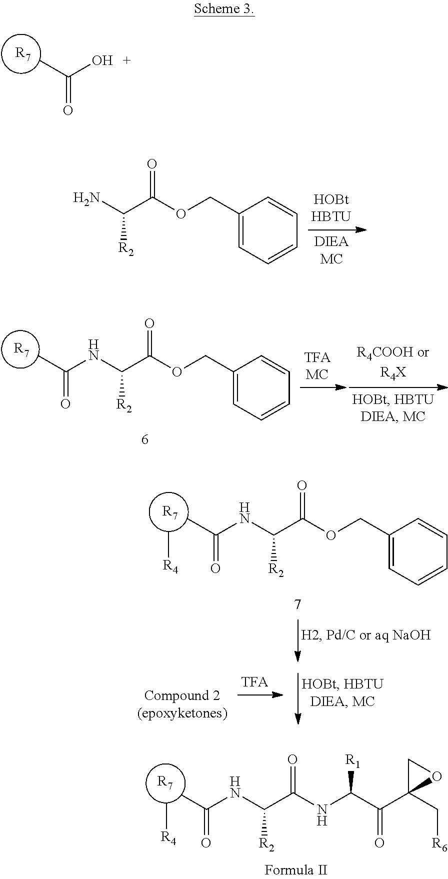

Synthesis of Formula III Compounds

Amide coupling or alkylation or acylation of amino acids using coupling agents (HOBt and HBTU) or organic base DIEA in methylene chloride furnished intermediate 8.

Finally, acid deprotected compounds 8 were coupled with Boc-deprotected epoxides (2) to generate the final compounds.

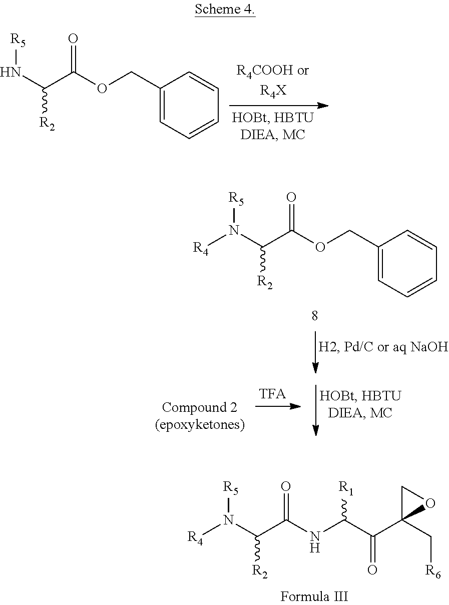

Synthesis of Formula IV Compounds

Dipeptide 9 was prepared by coupling two amino acids using coupling agents (HOBt and HBTU) in the presence of organic base DIEA. Deprotection of Boc followed by another peptide coupling reaction gave tripeptide 10. Removal of Boc from tripeptide 9 in the presence of TFA and methylene chloride and subsequent amide coupling or alkylation or acylation furnished peptide backbone 11.

Finally, acid-deprotected compounds 11 were coupled with Boc-deprotected epoxides (2) to generate the final compounds.

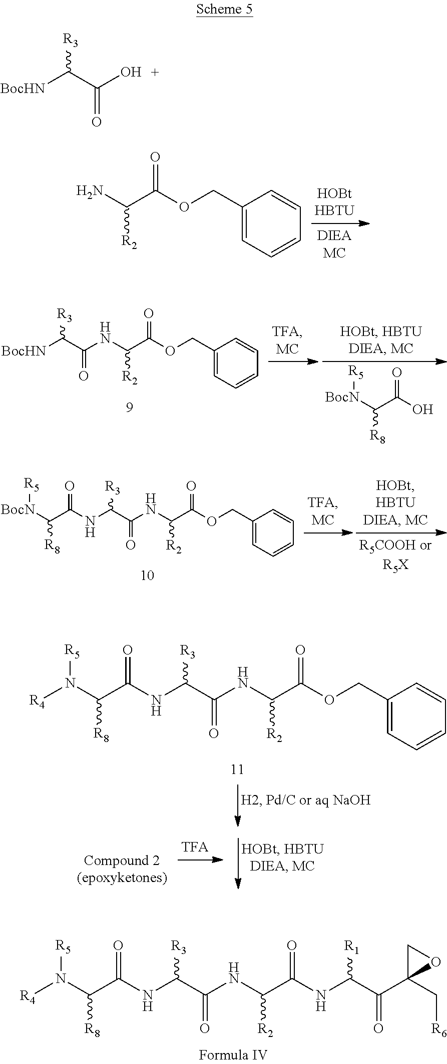

### Example 2: LMP2-Selective Inhibitors

As proof of concept for the utility of LMP2-selective inhibitors as a therapeutic target in autoimmune and neurodegenerative diseases, YU-102 was investigated.

Effect of YU102 on APPsw and LPS-Induced Memory Impairment:

To investigate the impacts of LMP2 inhibition on progressive memory and learning impairments, we have prepared LMP2-targeting compounds based on the structure of the previously reported YU102 (Table 2).

TABLE 2
Structures of LMP2 inhibitors.

| | |
|---|---|
| 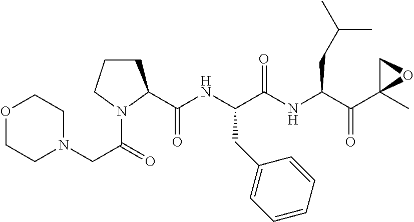 | 1 |
| 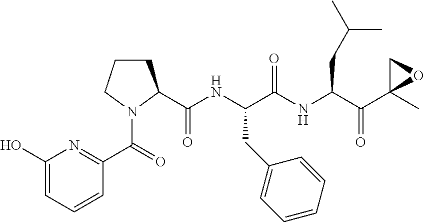 | 2 |
| 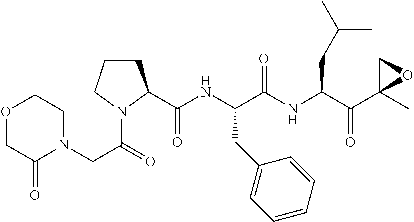 | 3 |
| 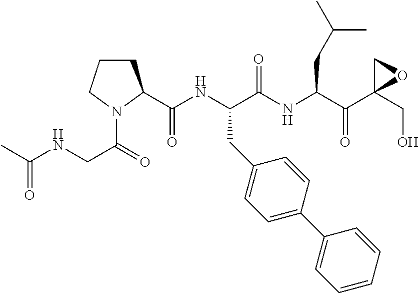 | 4 |
| 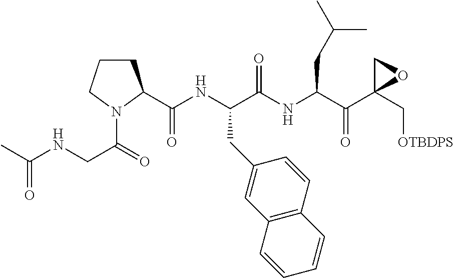 | 5 |
| 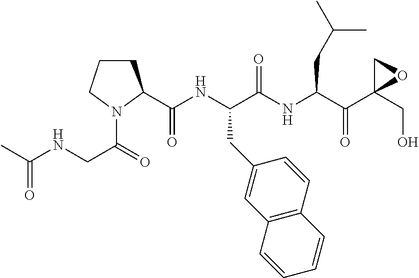 | 6 |
| 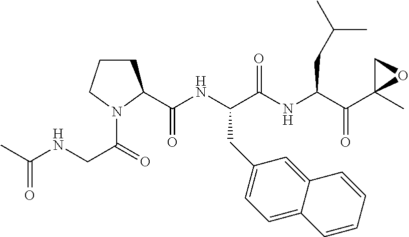 | 7 |
| 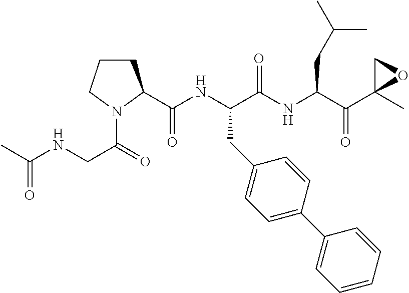 | 8 |
| 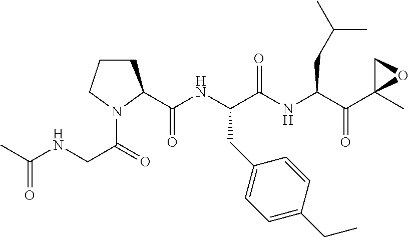 | 9 |
| 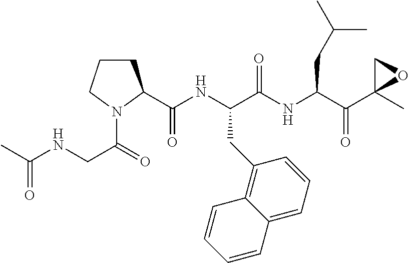 | 10 |
| 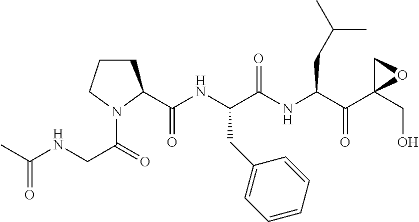 | 11 |
| 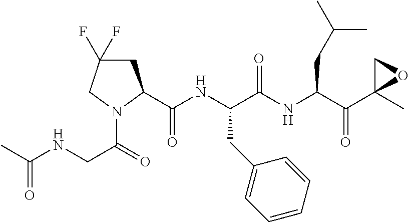 | 12 |
| 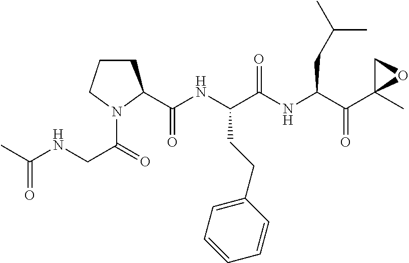 | 13 |
| 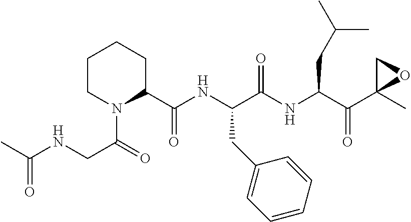 | 14 |
| 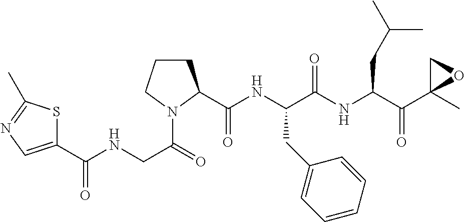 | 15 |
| 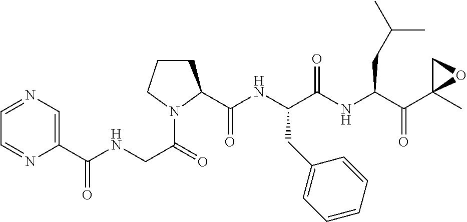 | 16 |
| 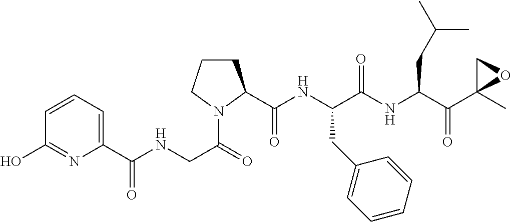 | 17 |
| 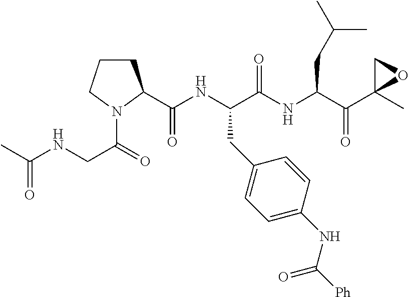 | 18 |
| 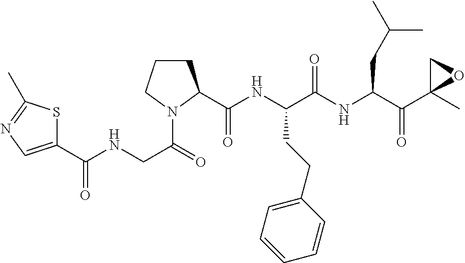 | 19 |
| 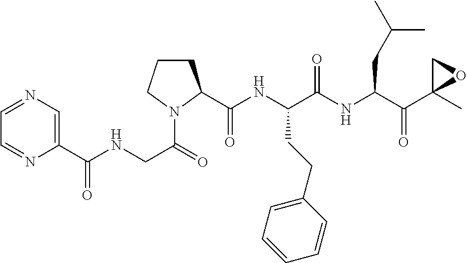 | 20 |
| 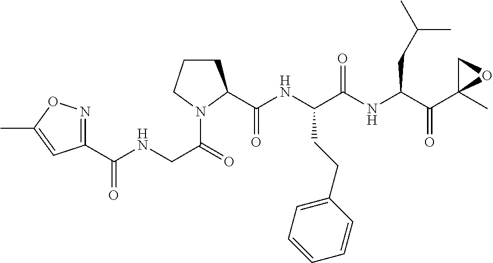 | 21 |
| 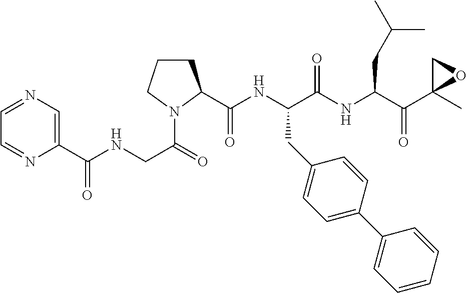 | 22 |
| 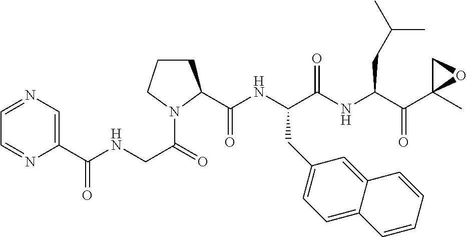 | 23 |
| 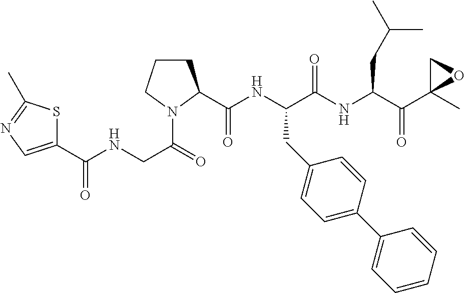 | 24 |
| 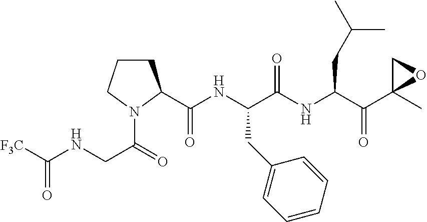 | 25 |
| 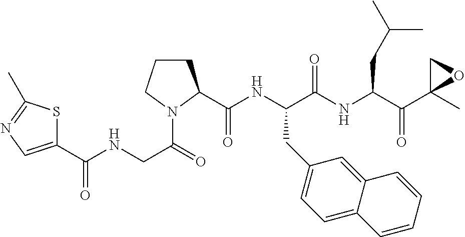 | 26 |
| 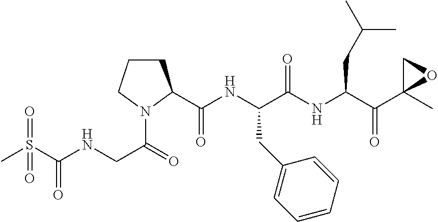 | 27 |
| 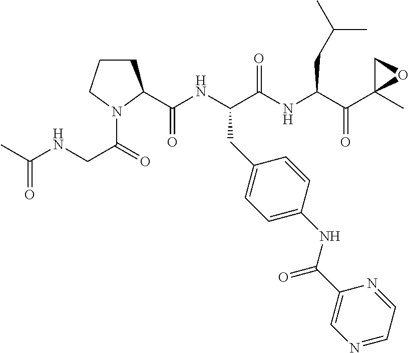 | 28 |
| 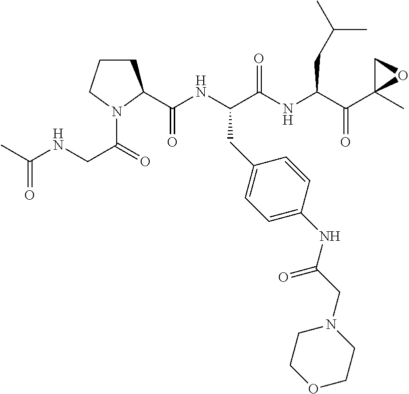 | 29 |
| 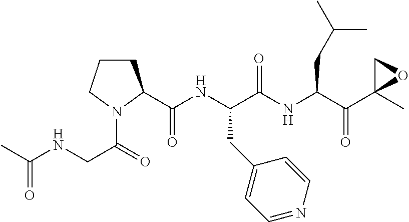 | 30 |
| 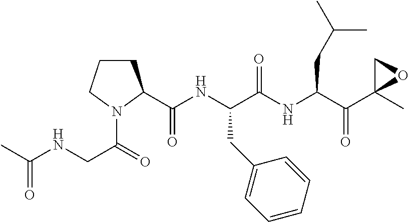 | YU102 |

After initial screening using purified 20S proteasomes (constitutive and immunoproteasome) (Table 2), two LMP2-selective inhibitors (YU102 & compound #16) were selected and further investigated for their LMP2 selectivity over other proteasome catalytic subunits (Table 3). These two compounds were used to investigate the impacts of LMP2 inhibition on cognitive deficits in two animal models of human Alzheimer's disease (AD). In these AD models, AD develops by Aβ deposition (APPsw transgenic mouse) or lipopolysaccharide (LPS)-induced neuroinflammation. For the APPsw transgenic model, also known as Tg2576, 10-month old APPsw mice were treated i.p. with LMP2 inhibitors twice a week for 3 weeks. For the LPS model, ˜8 week-old mice (ICR strain) were treated i.p. with YU102 twice a week for 3 weeks and then LPS daily for 5 days. Once treatments were finished, Morris water maze test was performed. All mice were trained for three times a day for 5 days before the test. The escape latency and distance traveled were recorded on a daily-basis over a 5-day period. Remarkably, as shown in FIGS. 8 and 12, mice treated with LMP2 inhibitors exhibited significantly shorter distance and escape latency compared to respective control groups. It should be noted that all mice tested did not display any irregularity in their motility. Mice treated with YU102 epimer, which is an inactive stereoisomer of YU102, showed similar escape latency and distance traveled to those of respective controls (FIG. 12). This supports that the efficacy of LMP2 inhibitors is mediated through LMP2 inhibition. One day after the completion of Morris water maze test, the ability of mouse to maintain memory was evaluated by probe trials. In line with the results from the water maze test, LMP2 inhibitor-treated mice performed significantly better than control groups: the percentage of time spent in the target quadrant was 21.25±2.71% for LMP2 inhibitor-treated group and ˜10-14.50±1.03% for control groups (FIGS. 8 & 12). Sequentially, a step-through latency test was performed a day after the probe trial. While APPsw and LPS-induced control groups showed an average step-through latency of ˜44 sec and ˜78 sec, respectively, LMP2 inhibitor-treated group had ˜128 sec, displaying considerably improved memory function (FIGS. 8 & 12).

Efficacy of YU102 in AD Models is Independent of Aβ Deposition.

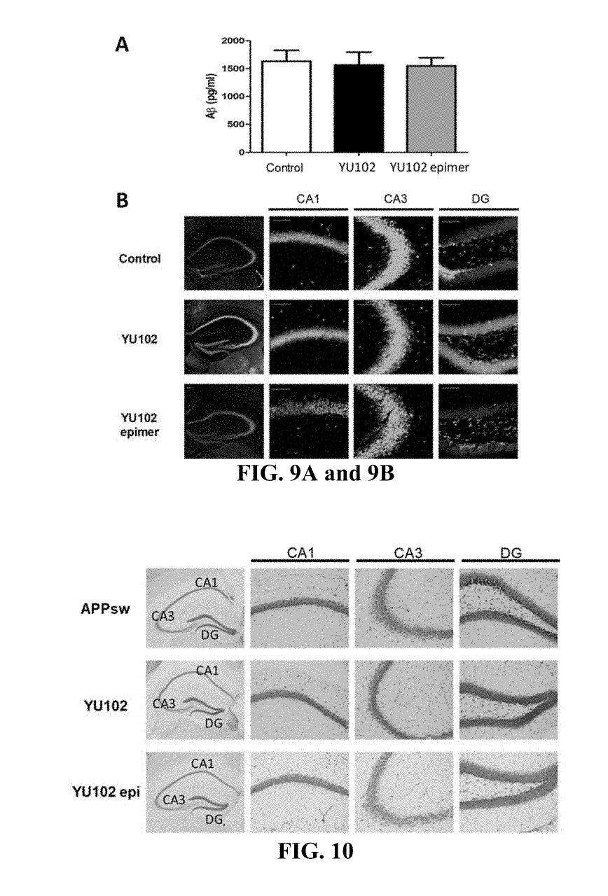

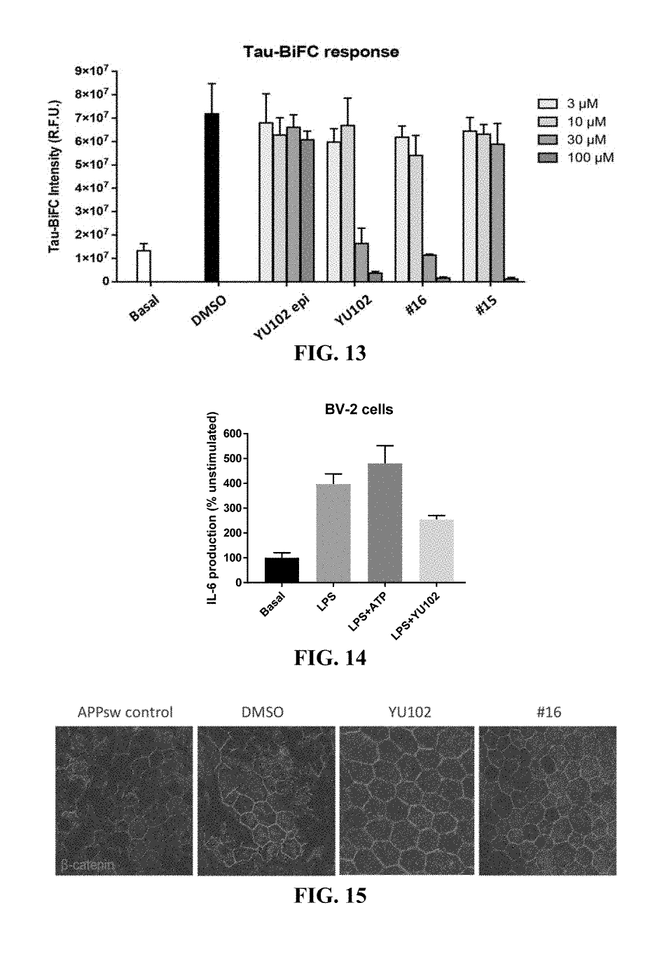

Given the data showing improved learning and memory of LMP2 inhibitor-treated mice and the role of Aβ deposition in memory dysfunction, we suspected that LMP2 inhibitors promote Aβ clearance in the brain of APPsw mice. To test this, we measured the expression levels of Aβ in hippocampal tissues isolated from the brains of APPsw mouse, using a ELISA-based assay and Thioflavin T (ThT) staining. To our surprise, we observed no difference in Aβ deposition between LMP2 inhibitor (YU102)-treated mouse and APPsw control (FIG. 9). Taken together, it appears that LMP2 inhibitors exert its anti-AD efficacy in these animal models independent of Aβ deposition. Furthermore, the result is highly intriguing in that Aβ-targeting drugs failed to demonstrate clinically meaningful efficacy in several high profile clinical trials. As a result, tau protein has become a prominent target for developing disease-modifying drugs. Given this, we wondered whether LMP2 inhibitors could block tau aggregation. To test this, we used HEK293-tau-BiFC cells to monitor the impacts of LMP2 inhibitors on tau aggregation. As shown in FIG. 13, LMP2 inhibitors did not inhibit tau aggregation. These results demonstrate that LMP2 inhibition attenuates disease progress in the APPsw mouse model of human AD independent of Aβ deposition and tau protein aggregation.

YU102 Displays No Neuroprotective Effects.

Since LMP2 inhibitors had no effects on Aβ or tau aggregation, we wondered if these compounds are simply protecting neurons from cell death caused by Aβ or LPS. To examine this, we performed Cresyl violet staining experiments on neuronal tissues isolated from the brains of APPsw mice. As shown in FIG. 10, YU102 displayed no neuroprotective effects, indicating that these LMP2 inhibitors do not function as neuroprotectants in this mouse model.

LMP2 Inhibitors Block Activation of Astrocytes and Microglia.

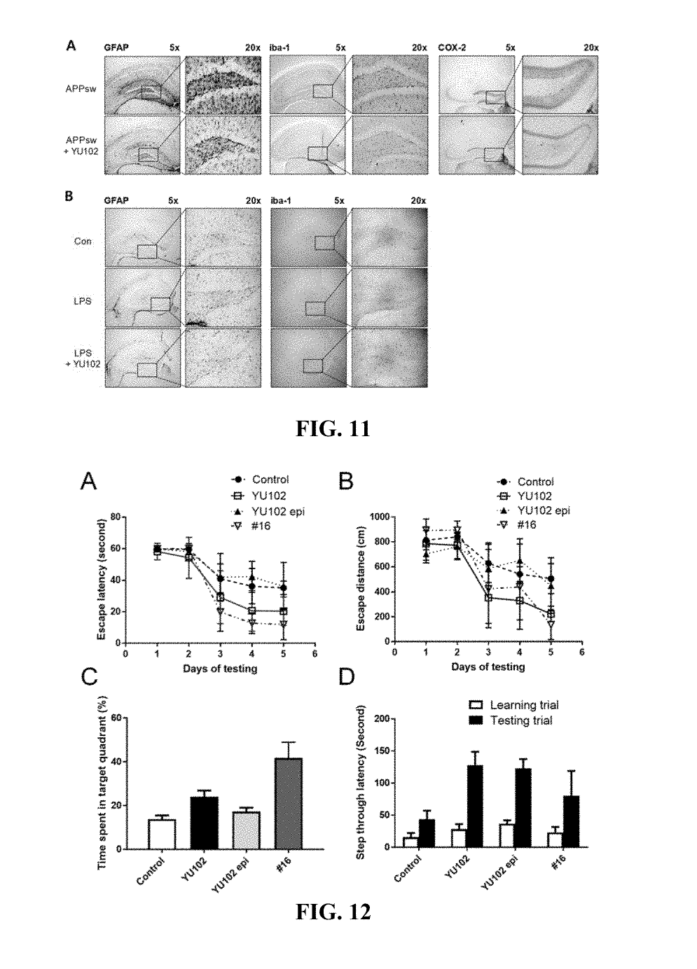

Activation of neuroglia is closely linked to neuroinflammation, which is reported to be one of major factors involved in the development and progression of AD. Therefore, we set out to investigate whether LMP2 inhibitors could block the activation of astrocytes and microglia. We performed immunohistochemical analysis of hippocampal tissues isolated from the brains of both APPsw and LPS-induced mice, using GFAP and Iba1 as standard markers for reactive astrocytes and microglial, respectively. As shown in FIG. 11, there were less reactive astrocytes and microglia in hippocampal tissues from AD mouse treated with LMP2 inhibitor compared to control APPsw mouse.

To directly verify the role of LMP2 in neuroinflammation pathways, we measured the expression of COX-2 by immunohistochemical analysis of hippocampal tissues from APPsw mouse. Our data confirmed that COX-2 is up-regulated in control mice in response to inflammatory insults and inhibition of LMP2 by YU102 significantly curtails COX-2 upregulation (Right panel, FIG. 11A).

LMP2 Inhibitors Attenuate Pro-Inflammatory Cytokine Production.

It has been reported that LPS increases proteasome activity and cytokine production in microglia and induces innate immune signaling and microglial activation.²⁵ Since LMP2 inhibitors reduced the number of activated microglia cells in APPsw mice, we suspected that they could suppress cytokine production. To test this, we used BV-2 cell line, a microglial cell line, known to upregulate cytokines in response to LPS treatment. As shown in FIG. 14, LMP2 inhibitors attenuate IL-6 production induced by LPS. Interestingly, a LMP7-selective inhibitor ONX0914 was less effective in blocking IL-6 production. This is highly interesting in that previous studies in human peripheral blood mononuclear cells (PBMCs) reported no effect of LMP2 inhibition on cytokine production.⁹ In the same studies, ONX0914 almost completely blocked LPS-induced cytokine production including IL-6. We suspect these contradictory results are due to cell type-specific role of LMP2, indicating a distinct role of LMP2 in microglia inflammatory response. Altogether, these findings demonstrate that inhibition of LMP2 ameliorates disease in mouse models of AD and may offer a promising strategy for AD treatment.

LMP2 Inhibitors Attenuate RPE (Retinal Pigment Epithelium) Degeneration in APPsw Mouse.

Several recent studies demonstrate that Aβ deposits are consistently found in the retina from many patients with age-related macular degeneration (AMD) and can be positively correlated with the disease progress.²⁶ Reports also show that patients with AD and animal models of human AD are more likely to suffer from AMD.²⁷⁻²⁹ Therefore, we investigated the effects of LMP2 inhibitors on retinal structure and photoreceptor mosaic integrity in APPsw mouse models of human AD. As shown in FIG. 15, LMP2 inhibitors protected the structural integrity of retina from degeneration. This suggests that LMP2 inhibitors could be used as a dual therapeutic agent targeting both AD and AMD.

Altogether, these findings demonstrate that inhibition of LMP2 significantly attenuates disease progression in mouse models of Alzheimer's disease independent of Aβ deposition and may offer a promising strategy for AD treatment.

Experimental:

Proteasome Activity Assay.

Purified 20S human proteasomes (CP and IP, R&D Systems) were used to assess the in vitro inhibition of proteasome catalytic activity by LMP2 inhibitors. In our 96-well format assays involving a 100 μL total volume, 20S proteasomes (0.5 μg/mL) were incubated with inhibitors or reference compounds (e.g., carfilzomib) in assay buffer (20 mM Tris-HCl, 0.5 mM EDTA, 0.035% SDS) at room temperature for 30 min. Reactions were initiated by the addition of fluorogenic substrates containing the AMC (7-amino-4-methylcoumarin) group. Specifically, the following substrates were used: Suc-LLVY-AMC (CT-L activity, 100 μM), Ac-PAL-AMC (LMP2, 100 μM), Ac-WLA-AMC (β5, 20 μM), Ac-nLPnLD-AMC (β1,100 μM), Ac-RLR-AMC (β2/2i, 20 μM), and Ac-ANW-AMC (β5i, 100 μM). The fluorescence of liberated AMC was measured over a period of 90 min at 360 and 460 nm on a SpectraMax M5 fluorescence plate reader (Molecular Devices). For the proteasome activity of brain tissues isolated from AD mice, tissues were homogenized in RIPA buffer (50 mM Tris Cl, pH 7.4, 150 mM NaCl, 5 mM EDTA, 1% Nonidet P-40, 1% sodium deoxycholate, 0.1% SDS, 1% aprotinin, 50 mM NaF) and sonicated. Samples were centrifuged for 40 min at 14,000×g (4° C.). Protein concentration was determined using the Bradford Assay according to the manufacturer's protocol. Finally, samples were loaded onto a 96-well plate followed by the addition of the substrate (Ac-PAL-AMC) at 37° C. Fluorescence was recorded for 90 min using a Synergy-HT (Bio Tek) plate reader. To exclude non-proteasomal substrate degradation, control samples were incubated with YU102 (1 μM) for 60 min at 37° C. before loading on the plate and values were subtracted from lysates incubated with DMSO control.

Immunohistochemical Staining.

Frozen tissues were cut into 30 μm thick sections and stored free floating in cryoprotectant solution (30% ethylenglycol, 20% glycerol, 50 mM sodium phosphate buffer, pH 7.4) at 4° C. until further use. For immunohistological staining, sections were rinsed in 1×PBS, incubated in blocking buffer (1×PBS containing 0.3% Triton X-100 and 10% normal goat serum) for 1 hr at room temperature and primary antibodies for GFAP (1:1000; Abcam) and Iba-1 (1:500; Abcam) were diluted in 1×PBS/0.3% triton X-100/5% normal goat serum and incubated over night at 4° C. Sections were washed with 1×PBS to wash off excessive primary antibodies, incubated with species specific peroxidase-coupled secondary antibodies (goat anti-mouse or goat anti-rabbit (1:300, Abcam)) diluted in 1×PBS/0.3% Triton X-100/5% normal goat serum and incubated for 1 h on a shaker at RT before developed with liquid diaminobezadine (DAB) (Dako, K3647). Sections were counterstained with matured hematoxylin, followed by dehydration in an ascending alcohol series before covered using Roti®-Histokitt II mounting medium. For Congo red staining, cerebral free floating sections were mounted on glass slides. Sections were incubated in stock solution I (0.5 M NaCl in 80% ethanol, 1% NaOH) for 20 min and in stock solution II (8.6 mM Congo red in stock solution I, 1% NaOH) for 45 min. After rinsing twice in absolute ethanol, sections were counterstained with mature hematoxylin and dehydrated in ascending alcohol series, twice rinsed in 98% xylene for 1 min, before mounting with Roti®-Histokitt II mounting medium. Light microscopy and stereology were performed using a Stereo Investigator system (MicroBrightField) and DV-47d camera (MicroBrightField) mounted on an Olympus BX53 microscope (Olympus, Germany). Fluorescence imaging was performed using an Olympus XM10 monochrome fluorescence CCD camera (Olympus, Germany).

Measurement of Aβ.

Aβ₁₋₄₂ levels were determined using specific ELISA Kit (Cusabio Biotech Co., Ltd., Wilmington, Del., USA). Experiments were performed according to each manufacturer's instructions. In brief, samples and standards were added into the pre-coated plate and incubated for 2 hours at 37° C. Biotin-Antibody (1×) was added to each well and incubated 1 hour at 37° C. After wash steps, HRP-avidin (1×) was incubated for 1 hour at 37° C. TMB substrate was added to each well after washing. The absorbance was measured at 450 nm using a microplate reader (Sunrise™, TECAN, Switzerland) after adding stop solution.

Thioflavin T Staining

Frozen brain tissues were cut into 30 μm sections by using cryostat microtome (Leica CM1850; Leica Microsystems). The pieces of brain were thoroughly washed with distilled water for 5 min, and then transferred to gelatin coated slides and placed in 1% Thioflavine T for 5 min, followed by dehydration using ascending grades of ethanol (50%, 70%, 90%, and 100%) for 2 min. The dehydrated samples were then mounted with mounting medium (Fluoromount™, Sigma). The Thioflavin T staining was examined by using a fluorescence microscope.

Cresyl Violet Staining.

Frozen brain tissues were cut into 30 μm sections by using cryostat microtome (Leica CM1850; Leica Microsystems, Korea). The pieces of brain were thoroughly washed with PBS to remove the excess fixative agent, and then transferred to gelatin coated slices and stained with 0.1% Cresyl violet (2-5 minutes) to identify cortical layers and cytoarchitectural features of isocortical region. Next, the sections were washed with distill water and dehydrated by using ascending grades of ethanol (50%, 70%, 90%, and 100%) for 2 min in each grade followed by a 1-min immersion in a 1:1 mixture of absolute alcohol and xylene. The sections were then rinsed with xylene for 5-10 min and mounted with mounting medium (CYTOSEAL™ XYL; Thermo Scientific, USA). The tissue was photographed (100×) in the same areas.

Microglia Cell Isolation and Stimulation.

For cell isolation procedure, the whole brain was placed in HBSS on ice and further processed with neuronal kit dissociation according to manufacturer's instructions. Isolation of CD11b+ cells from brain tissue was performed using the Neural Tissue Dissociation Kit (P) (Miltenyi Biotech) and the magnetic cell sorting (MACS) technique using CD11b-labeled magnetic Microbeads (Miltenyi Biotech) according to manufacturer's instructions. 50,000 CD11b+ microglia were cultured overnight in a 96 well plate. Culture medium was removed and LPS (1 μg/ml) diluted in serum-free culture medium was added to the wells. Equivalent amount of PBS in serum-free medium was used as stimulation negative control. Supernatant for baseline measurements were collected prior to LPS stimulation. After 24 h, medium was collected, snap frozen in liquid nitrogen and stored at −80° C. for further cytokine analysis as described above.

Behavioral Analysis.

The Morris water maze test is a widely accepted method for examining cognitive function and performed as described previously.³⁰ Briefly, a circular plastic pool was filled with water maintained at 22-25° C. An escape platform was submerged 1-1.5 cm below the surface of the water.

The learning trials were conducted over 5 days, with 4 trials per day, with three randomized starting points. The position of the escape platform was kept constant. Each trial lasted for 60 s or ended as soon as the mice reached the submerged platform. Swimming pattern of each mouse was monitored and recorded by a camera mounted above the center of the pool, and the escape latency, escape distance and swimming speed were assessed by the SMART-LD program (Panlab, Spain). A quiet environment and constant water temperature were maintained throughout the experimental period.

To assess memory consolidation, a probe test was performed 48 hr after the water maze test (i.e. Day 7). For the probe test, the platform was removed from the pool and the mice were allowed to swim freely. The swimming pattern of each mouse was monitored and recorded for 60 s using the SMART-LD program (Panlab). Consolidated spatial memory was estimated by the time spent in the target quadrant area.

The passive avoidance response was determined using a “step-through” apparatus (Med Associates, USA). The probe test (i.e. Day 9) was performed a 48 hr after training trials. Specifically, each mouse was placed in a illuminated compartment of the apparatus facing away from the dark compartment. There is a small opening in the dark insert that allows the mouse to freely move between the two zones. When the mouse fully exits the bright compartment and enters the dark compartment, it receives an electric shock (0.4 mA, 3 s duration). Each mouse was placed in the illuminated compartment 24 hr after the training trial (i.e. Day 10) and the latency to enter the dark compartment was measured, with a 180-sec cut-off time.

Tau Protein Dimerization Detection Assay.

For microscopic image analysis, cells were plated in a black transparent 96-well plate. The next day, tau-BiFC cells were treated with the okadaic acid or forskolin at various concentrations. After, 2, 9, 19, and 24 hrs of incubation, the entire 96-well plate was automatically imaged under same exposure by using Operetta® High Contents Screening System (equipped with a 10× and 20× dry lenses). The cellular intensities of tau-BiFC fluorescence were analyzed using Harmony 3.1 software. Error bars indicate s.d. from two independent experiments. Each experiment was performed as triplicate.

TABLE 3
Inhibitory potency of proteasome inhibitors toward the immunoproteasome subunits.

YU102

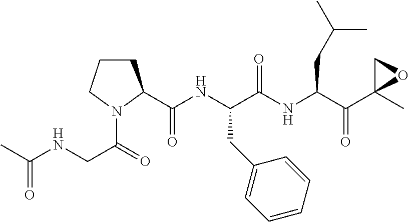

16

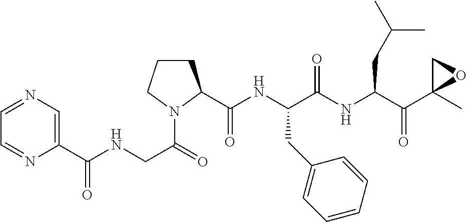

ONX0914

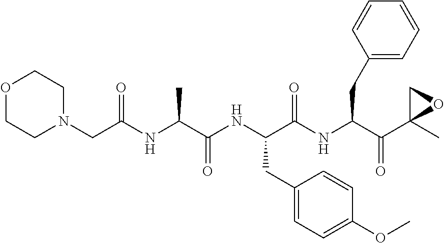

IC₅₀ (nM)

| Proteasome | | | | LMP7/ |
|---|---|---|---|---|
| inhibitors | LMP2/β1i | Y/β1 | X/β5 | β5i |
| YU102 | 105.2 | 206.7 (1.93x) | >10000 | >10000 |
| 16 | 70.0 | 589.9 (8.42x) | >10000 | >10000 |
| ONX-0914 | 1009 | >10000 | 472.7 | 37.9 |

### CONCLUSIONS

The present inventors have successfully developed a group of proteasome-inhibiting peptide epoxyketone compositions, including proteasome subunit LMP2 inhibitors and general proteasome inhibitors. The inhibitors utilize a peptide epoxyketone scaffold with modifications at the P1′ position to mediate proteasome inhibition. In addition to its unique scaffold, the compounds show effectiveness in models of proteasome inhibitor resistance. This is notable, as MM patients who are initially responsive to currently FDA-approved proteasome inhibitors almost inevitably develop resistance to those drugs. Therefore, these unique compounds provide an opportunity for an additional option for these refractory MM patients. Multiple clinical trials clearly demonstrated that the clinically approved proteasome inhibitors carfilzomib and bortezomib lack utility in the treatment of solid tumors due to their rapid metabolism, irreversible inhibition, sensitivity to resistance, and dose-limiting toxicities.^{18,20,22,41,42}

The immunoproteasome is an inducible proteasome variant containing immuno-subunits low-molecular mass polypeptide-7 (LMP7), LMP2 and multicatalytic endopeptidase complex-1 (MECL-1) in place of constitutive counterparts X, Y and Z, respectively. While previous studies have found that the immunoproteasome is up-regulated in the brains of Alzheimer's disease (AD) patients, the exact role of the immunoproteasome in AD remains poorly defined. As disclosed herein, the impacts of LMP2 inhibition are characterized on progressive memory and learning impairments caused by amyloid-β (Aβ) deposition or lipopolysaccharide (LPS)-induced neuroinflammation in mouse models. Selective LMP2 inhibitors reversed disease progression independent of Aβ deposition or microglia activation. Data from these studies indicate that selective inhibition of LMP2 suppresses NLRP3 inflammasome-mediated neuroinflammation (neurotrauma) to improve the pathophysiology of neurodegenerative conditions in animal models. The impacts of LMP2 inhibition on progressive memory and learning impairments caused by amyloid-β (Aβ) deposition or lipopolysaccharide (LPS)-induced neuroinflammation was further explored in mouse models. Selective LMP2 inhibitors reversed disease progression independent of Aβ deposition and tau aggregation. Data from the studies indicate that selective inhibition of LMP2 suppresses cytokine production such as IL-6 and ameliorates neurodegenerative phenotypes in animal models. Overall, these findings suggest a role for LMP2 in the regulation of neuroinflammation and support that inhibition of LMP2 may offer a unique strategy for neurodegenerative diseases such as AD.

Throughout this document, various references are mentioned. All such references are incorporated herein by reference, including the references set forth in the following list:

### REFERENCES

1. Muchamuel, T.; Basler, M.; Aujay, M. A.; Suzuki, E.; Kalim, K. W.; Lauer, C.; Sylvain, C.; Ring, E. R.; Shields, J.; Jiang, J.; Shwonek, P.; Parlati, F.; Demo, S. D.; Bennett, M. K.; Kirk, C. J.; Groettrup, M. A selective inhibitor of the immunoproteasome subunit LMP7 blocks cytokine production and attenuates progression of experimental arthritis. Nat Med 2009, 15, (7), 781-7.
2. Chen, X.; Zhang, X.; Wang, Y.; Lei, H.; Su, H.; Zeng, J.; Pei, Z.; Huang, R. Inhibition of immunoproteasome reduces infarction volume and attenuates inflammatory reaction in a rat model of ischemic stroke. Cell Death Dis 2015, 6, e1626.
3. Johnson, H. W. B.; Anderl, J. L.; Bradley, E. K.; Bui, J.; Jones, J.; Arastu-Kapur, S.; Kelly, L. M.; Lowe, E.; Moebius, D. C.; Muchamuel, T.; Kirk, C.; Wang, Z.; McMinn, D. Discovery of Highly Selective Inhibitors of the Immunoproteasome Low Molecular Mass Polypeptide 2 (LMP2) Subunit. ACS Med Chem Lett 2017, 8, (4), 413-417.
4. Jang, E. R.; Lee, N. R.; Han, S.; Wu, Y.; Sharma, L. K.; Carmony, K. C.; Marks, J.; Lee, D. M.; Ban, J. O.; Wehenkel, M.; Hong, J. T.; Kim, K. B.; Lee, W. Revisiting the role of the immunoproteasome in the activation of the canonical NF-kappaB pathway. Mol Biosyst 2012, 8, (9), 2295-302.
5. Keller, J. N.; Hanni, K. B.; Markesbery, W. R. Impaired proteasome function in Alzheimer's disease. J Neurochem 2000, 75, (1), 436-9.
6. Oh, S.; Hong, H. S.; Hwang, E.; Sim, H. J.; Lee, W.; Shin, S. J.; Mook-Jung, I. Amyloid peptide attenuates the proteasome activity in neuronal cells. Mech Ageing Dev 2005, 126, (12), 1292-9.
7. Tseng, B. P.; Green, K. N.; Chan, J. L.; Blurton-Jones, M.; LaFerla, F. M. Abeta inhibits the proteasome and enhances amyloid and tau accumulation. Neurobiol Aging 2008, 29, (11), 1607-18.
8. Almeida, C. G.; Takahashi, R. H.; Gouras, G. K. Beta-amyloid accumulation impairs multivesicular body sorting by inhibiting the ubiquitin-proteasome system. J Neurosci 2006, 26, (16), 4277-88.
9. Morozov, A. V.; Kulikova, A. A.; Astakhova, T. M.; Mitkevich, V. A.; Burnysheva, K. M.; Adzhubei, A. A.; Erokhov, P. A.; Evgen'ev, M. B.; Sharova, N. P.; Karpov, V. L.; Makarov, A. A. Amyloid-beta Increases Activity of Proteasomes Capped with 19S and 11S Regulators. J Alzheimers Dis 2016, 54, (2), 763-76.
10. Myeku, N.; Clelland, C. L.; Emrani, S.; Kukushkin, N. V.; Yu, W. H.; Goldberg, A. L.; Duff, K. E. Tau-driven 26S proteasome impairment and cognitive dysfunction can be prevented early in disease by activating cAMP-PKA signaling. Nat Med 2016, 22, (1), 46-53.
11. Orre, M.; Kamphuis, W.; Dooves, S.; Kooijman, L.; Chan, E. T.; Kirk, C. J.; Dimayuga Smith, V.; Koot, S.; Mamber, C.; Jansen, A. H.; Ovaa, H.; Hol, E. M. Reactive glia show increased immunoproteasome activity in Alzheimer's disease. Brain 2013, 136, (Pt 5), 1415-31.
12. Moritz, K. E.; McCormack, N. M.; Abera, M. B.; Viollet, C.; Yauger, Y. J.; Sukumar, G.; Dalgard, C. L.; Burnett, B. G. The role of the immunoproteasome in interferon-gamma-mediated microglial activation. Scientific reports 2017, 7, (1), 9365.
13. Aso, E.; Lomoio, S.; Lopez-Gonzalez, I.; Joda, L.; Carmona, M.; Fernandez-Yague, N.; Moreno, J.; Juves, S.; Pujol, A.; Pamplona, R.; Portero-Otin, M.; Martin, V.; Diaz, M.; Ferrer, I. Amyloid generation and dysfunctional immunoproteasome activation with disease progression in animal model of familial Alzheimer's disease. Brain Pathol 2012, 22, (5), 636-53.
14. Wagner, L. K.; Gilling, K. E.; Schormann, E.; Kloetzel, P. M.; Heppner, F. L.; Kruger, E.; Prokop, S. Immunoproteasome deficiency alters microglial cytokine response and improves cognitive deficits in Alzheimer's disease-like APPPS1 mice. Acta Neuropathol Commun 2017, 5, (1), 52.
15. Mundt, S.; Engelhardt, B.; Kirk, C. J.; Groettrup, M.; Basler, M. Inhibition and deficiency of the immunoproteasome subunit LMP7 attenuates LCMV-induced meningitis. Eur J Immunol 2016, 46, (1), 104-13.
16. Mishto, M.; Raza, M. L.; de Biase, D.; Ravizza, T.; Vasuri, F.; Martucci, M.; Keller, C.; Bellavista, E.; Buchholz, T. J.; Kloetzel, P. M.; Pession, A.; Vezzani, A.; Heinemann, U. The immunoproteasome beta5i subunit is a key contributor to ictogenesis in a rat model of chronic epilepsy. Brain Behav Immun 2015, 49, 188-96.
17. Mishto, M.; Bellavista, E.; Santoro, A.; Stolzing, A.; Ligorio, C.; Nacmias, B.; Spazzafumo, L.; Chiappelli, M.; Licastro, F.; Sorbi, S.; Pession, A.; Ohm, T.; Grune, T.; Franceschi, C. Immunoproteasome and LMP2 polymorphism in aged and Alzheimer's disease brains. Neurobiol Aging 2006, 27, (1), 54-66.
18. Mishto, M.; Bonafe, M.; Salvioli, S.; Olivieri, F.; Franceschi, C. Age dependent impact of LMP polymorphisms on TNFalpha-induced apoptosis in human peripheral blood mononuclear cells. Exp Gerontol 2002, 37, (2-3), 301-8.
19. Sacks, C. A.; Avorn, J.; Kesselheim, A. S. N Engl J Med 2017, 376, 1706-1708. Cummings, J.; Lee, G.; Mortsdorf, T.; Ritter, A.; Zhong, K. Alzheimers Dement (N Y) 2017, 3, 367-384.
20. Wise, J. BMJ 2016, 354, i4474.
21. Rafii, M. S. Lancet 2016, 388, 2842-2844.
22. Seifert, U.; Bialy, L. P.; Ebstein, F.; Bech-Otschir, D.; Voigt, A.; Schroter, F.; Prozorovski, T.; Lange, N.; Steffen, J.; Rieger, M.; Kuckelkorn, U.; Aktas, O.; Kloetzel, P. M.; Kruger, E. Cell 2010, 142, 613-24.
23. Kincaid, E. Z.; Che, J. W.; York, I.; Escobar, H.; Reyes-Vargas, E.; Delgado, J. C.; Welsh, R. M.; Karow, M. L.; Murphy, A. J.; Valenzuela, D. M.; Yancopoulos, G. D.; Rock, K. L. Nat Immunol 2011, 13, 129-35.
24. Opitz, E.; Koch, A.; Klingel, K.; Schmidt, F.; Prokop, S.; Rahnefeld, A.; Sauter, M.; Heppner, F. L.; Volker, U.; Kandolf, R.; Kuckelkorn, U.; Stangl, K.; Kruger, E.; Kloetzel, P. M.; Voigt, A. PLoS Pathog 2011, 7, e1002233.
25. Muchamuel, T.; Basler, M.; Aujay, M. A.; Suzuki, E.; Kalim, K. W.; Lauer, C.; Sylvain, C.; Ring, E. R.; Shields, J.; Jiang, J.; Shwonek, P.; Parlati, F.; Demo, S. D.; Bennett, M. K.; Kirk, C. J.; Groettrup, M. Nat Med 2009, 15, 781-7.
26. Johnson, H. W. B.; Anderl, J. L.; Bradley, E. K.; Bui, J.; Jones, J.; Arastu-Kapur, S.; Kelly, L. M.; Lowe, E.; Moebius, D. C.; Muchamuel, T.; Kirk, C.; Wang, Z.; McMinn, D. ACS Med Chem Lett 2017, 8, 413-417.
27. Jang, E. R.; Lee, N. R.; Han, S.; Wu, Y.; Sharma, L. K.; Carmony, K. C.; Marks, J.; Lee, D. M.; Ban, J. O.; Wehenkel, M.; Hong, J. T.; Kim, K. B.; Lee, W. Mol Biosyst 2012, 8, 2295-302.
28. Keller, J. N.; Hanni, K. B.; Markesbery, W. R. J Neurochem 2000, 75, 436-9.
29. Oh, S.; Hong, H. S.; Hwang, E.; Sim, H. J.; Lee, W.; Shin, S. J.; Mook-Jung, I. Mech Ageing Dev 2005, 126, 1292-9.
30. Tseng, B. P.; Green, K. N.; Chan, J. L.; Blurton-Jones, M.; LaFerla, F. M. Neurobiol Aging 2008, 29, 1607-18.
31. Almeida, C. G.; Takahashi, R. H.; Gouras, G. K. J Neurosci 2006, 26, 4277-88.
32. Myeku, N.; Clelland, C. L.; Emrani, S.; Kukushkin, N. V.; Yu, W. H.; Goldberg, A. L.; Duff, K. E. Nat Med 2016, 22, 46-53.
33. Orre, M.; Kamphuis, W.; Dooves, S.; Kooijman, L.; Chan, E. T.; Kirk, C. J.; Dimayuga Smith; Koot, S.; Mamber, C.; Jansen, A. H.; Ovaa, H.; Hol, E. M. Brain 2013, 136, 1415-31.
34. Moritz, K. E.; McCormack, N. M.; Abera, M. B.; Viollet, C.; Yauger, Y. J.; Sukumar, G.; Dalgard, C. L.; Burnett, B. G. Scientific reports 2017, 7, 9365.
35. Mishto, M.; Bellavista, E.; Santoro, A.; Stolzing, A.; Ligorio, C.; Nacmias, B.; Spazzafumo, L.; Chiappelli, M.; Licastro, F.; Sorbi, S.; Pession, A.; Ohm, T.; Grune, T.; Franceschi, C. Neurobiol Aging 2006, 27, 54-66.
36. Nijholt, D. A.; de Graaf, T. R.; van Haastert, E. S.; Oliveira, A. O.; Berkers, C. R.; Zwart, R.; Ovaa, H.; Baas, F.; Hoozemans, J. J.; Scheper, W. Cell Death Differ 2011, 18, 1071-81.
37. Aso, E.; Lomoio, S.; Lopez-Gonzalez, I.; Joda, L.; Carmona, M.; Fernandez-Yague, N.; Moreno, J.; Juves, S.; Pujol, A.; Pamplona, R.; Portero-Otin, M.; Martin, V.; Diaz, M.; Ferrer, I. Brain Pathol 2012, 22, 636-53.
38. Wagner, L. K.; Gilling, K. E.; Schormann, E.; Kloetzel, P. M.; Heppner, F. L.; Kruger, E.; Prokop, S. Acta Neuropathol Commun 2017, 5, 52.
39. Ho, Y. K.; Bargagna-Mohan, P.; Mohan, R.; Kim, K. B. Chem Biol 2007, 14, 419-430.
40. Mishto, M.; Bonafe, M.; Salvioli, S.; Olivieri, F.; Franceschi, C. Exp Gerontol 2002, 37, 301-8.
41. Tak, H.; Hague, M. M.; Kim, M. J.; Lee, J. H.; Baik, J. H.; Kim, Y.; Kim, D. J.; Grailhe, R.; Kim, Y. K. PLoS One 2013, 8, e81682.
42. Jiang, Z.; Georgel, P.; Du, X.; Shamel, L.; Sovath, S.; Mudd, S.; Huber, M.; Kalis, C.; Keck, S.; Galanos, C.; Freudenberg, M.; Beutler, B. Nat Immunol 2005, 6, 565-70.
43. Ratnayaka, J. A.; Serpell, L. C.; Lotery, A. J. Eye (Lond) 2015, 29, 1013-26.
44. Keenan, T. D.; Goldacre, R.; Goldacre, M. J. JAMA Ophthalmol 2014, 132, 63-8. Chiu, K.; Chan, T. F.; Wu, A.; Leung, I. Y.; So, K. F.; Chang, R. C. Age (Dordr) 2012, 34, 633-49.
45. Ding, J. D.; Lin, J.; Mace, B. E.; Herrmann, R.; Sullivan, P.; Bowes Rickman, C. Vision Res 2008, 48, 339-45.
46. Voorhees, C. V.; Williams, M. T. Nat Protoc 2006, 1, 848-58.

All publications, patents, and patent applications mentioned in this specification are herein incorporated by reference to the same extent as if each individual publication, patent, or patent application was specifically and individually indicated to be incorporated by reference.

It will be understood that various details of the presently disclosed subject matter can be changed without departing from the scope of the subject matter disclosed herein. Furthermore, the foregoing description is for the purpose of illustration only, and not for the purpose of limitation.

I claim:

1. A compound, a pharmaceutically acceptable salt, solvate, or physiologically functional derivative thereof, of the formula: 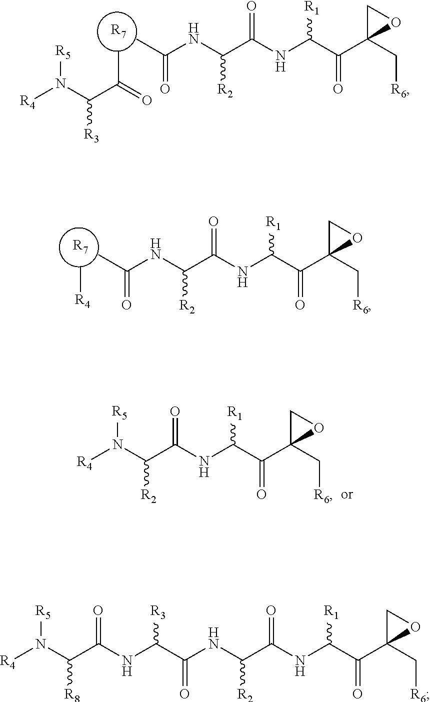
wherein R₁, R₂, R₃ and R₈ are independently selected from 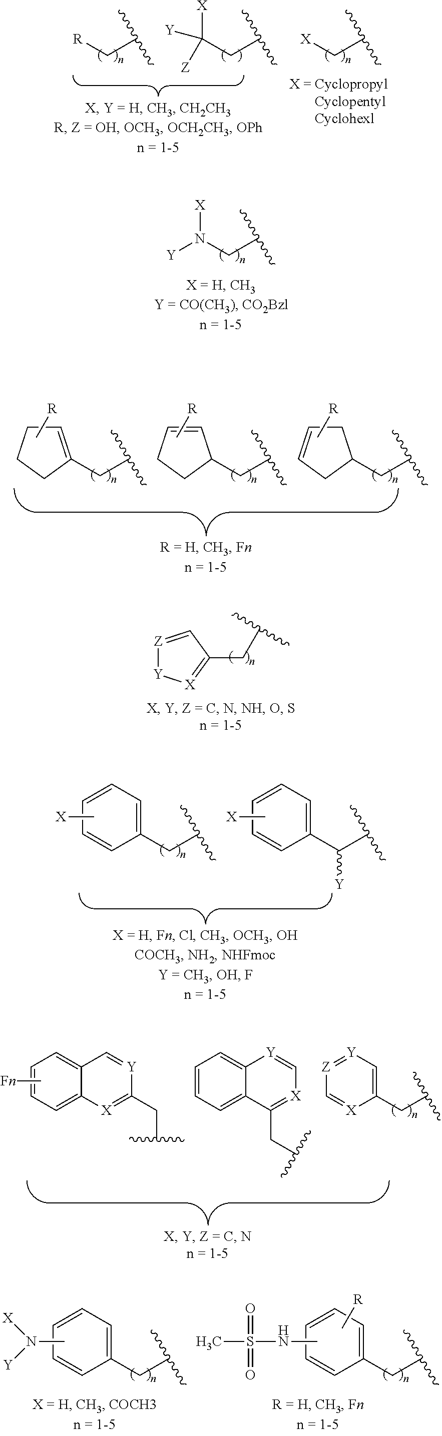 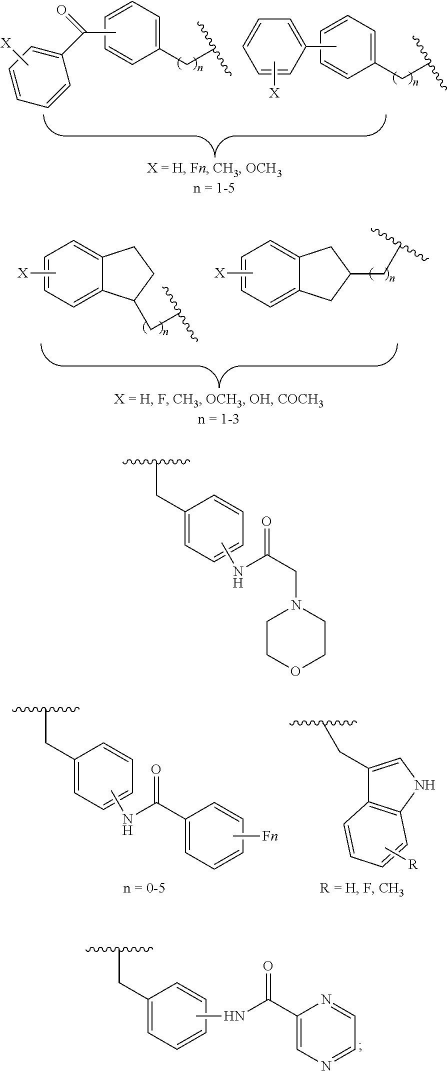
R₄ is selected from 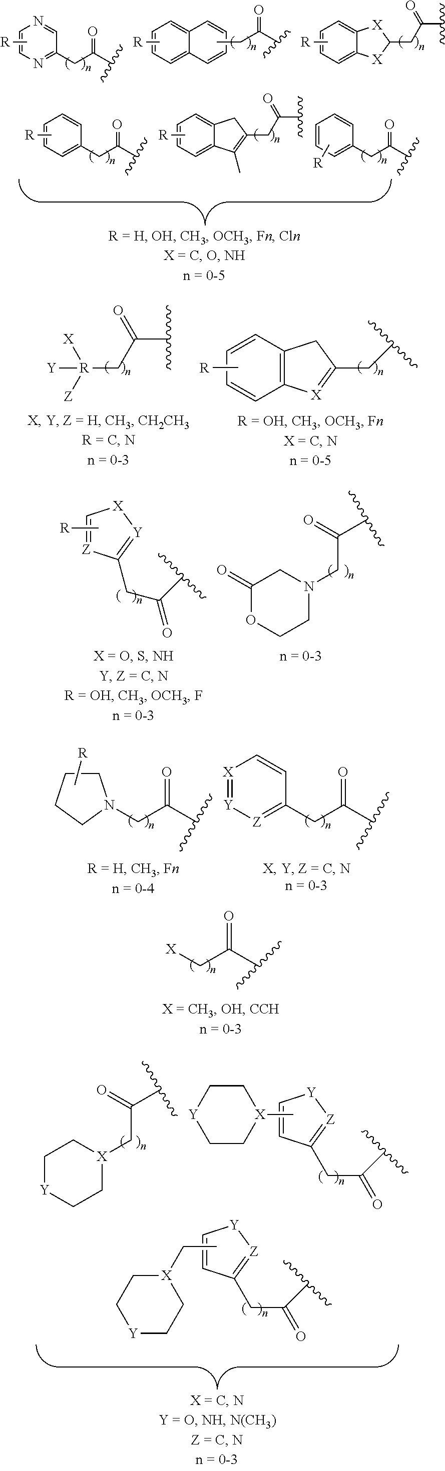 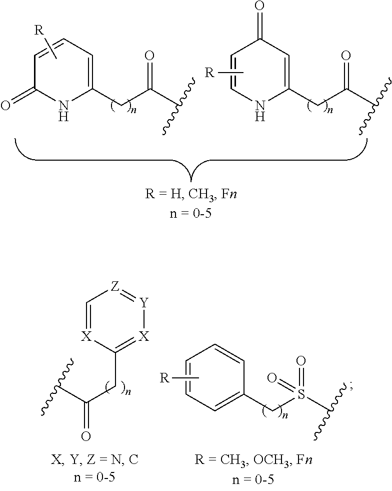
R₅ is selected from H or CH₃;
R₆ is selected from 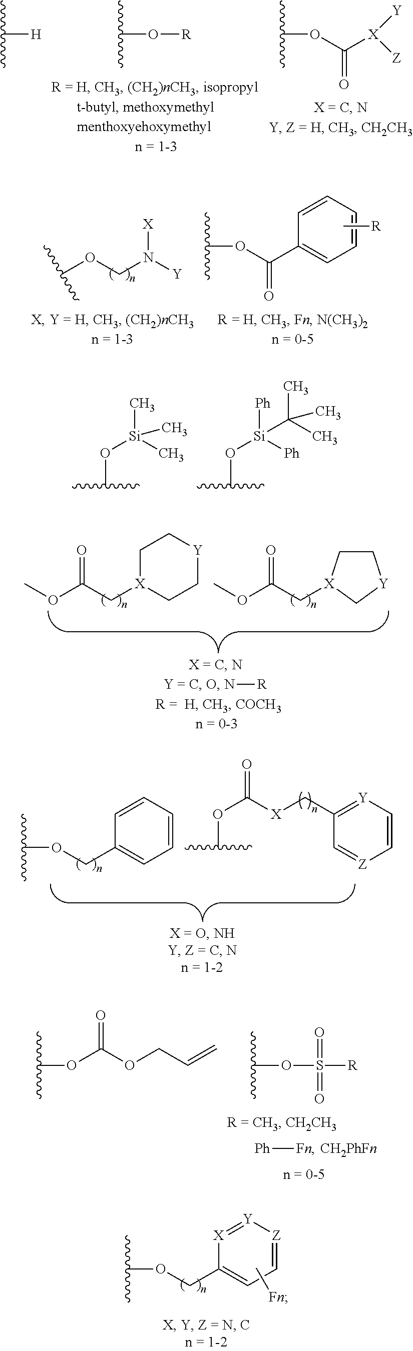
and R₇ is selected from

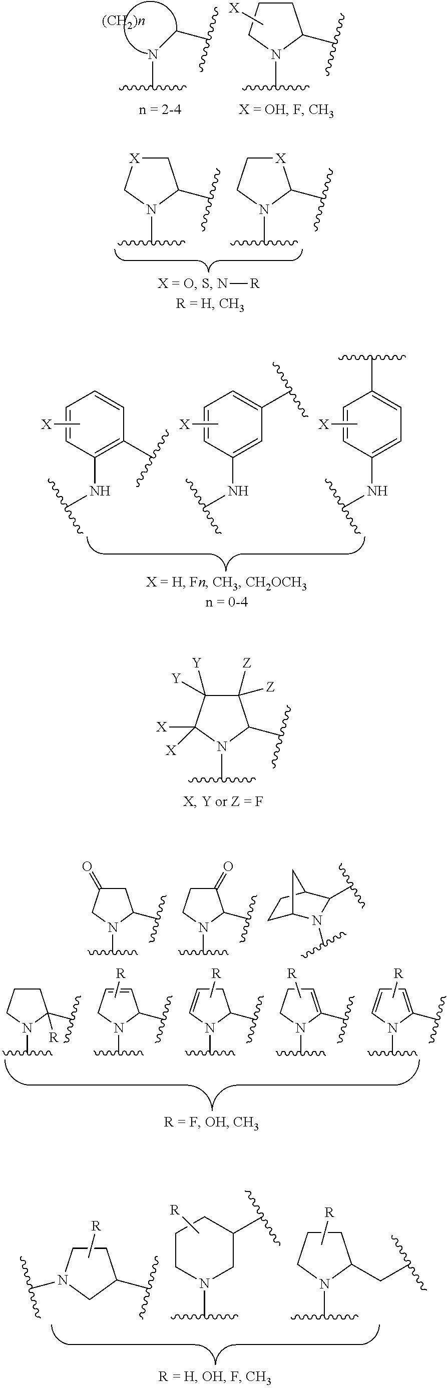

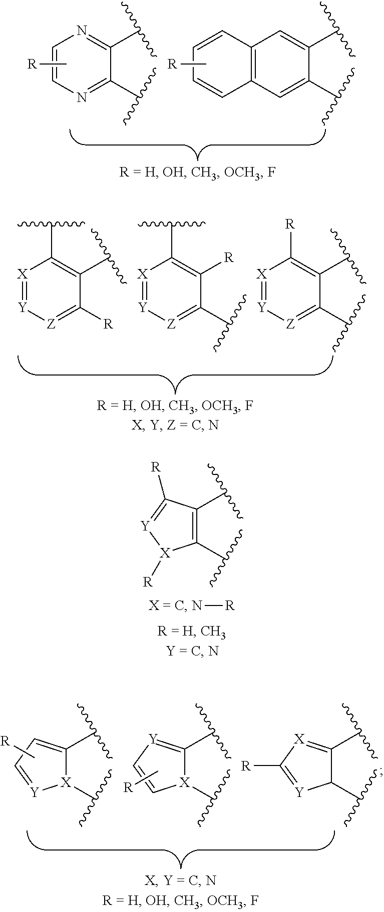

2. A compound, or a pharmaceutically acceptable salt, solvate, or physiologically functional derivative thereof selected from

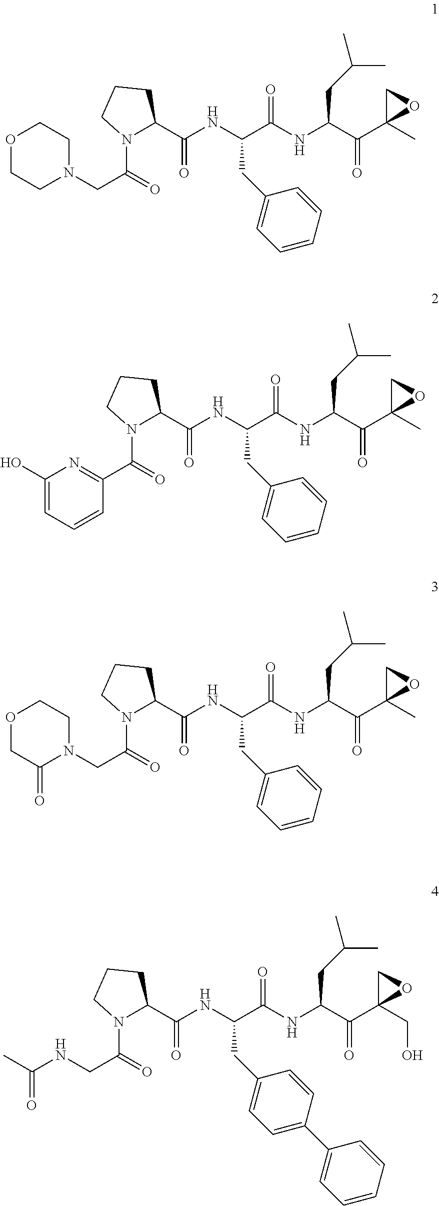

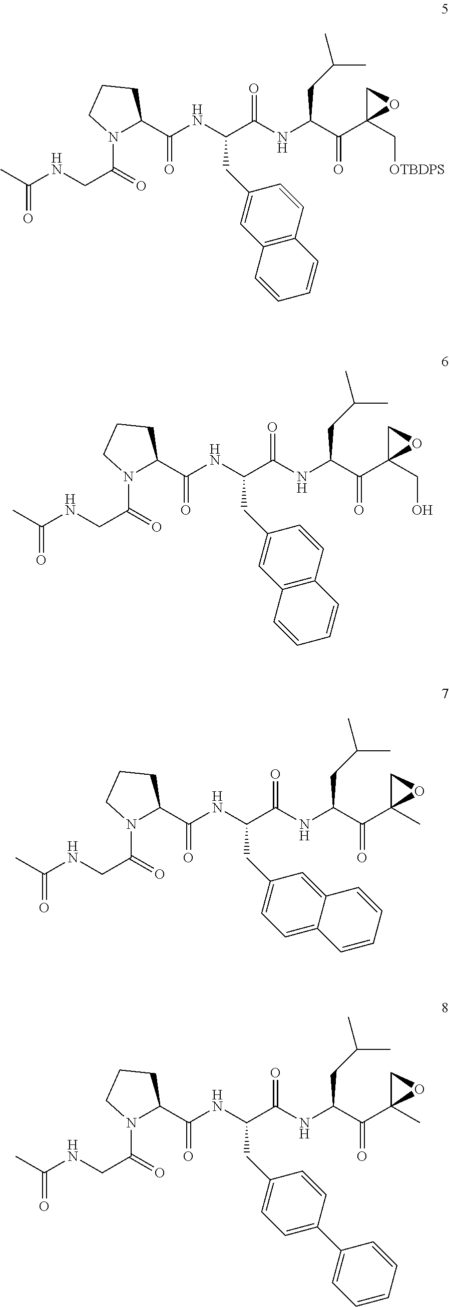

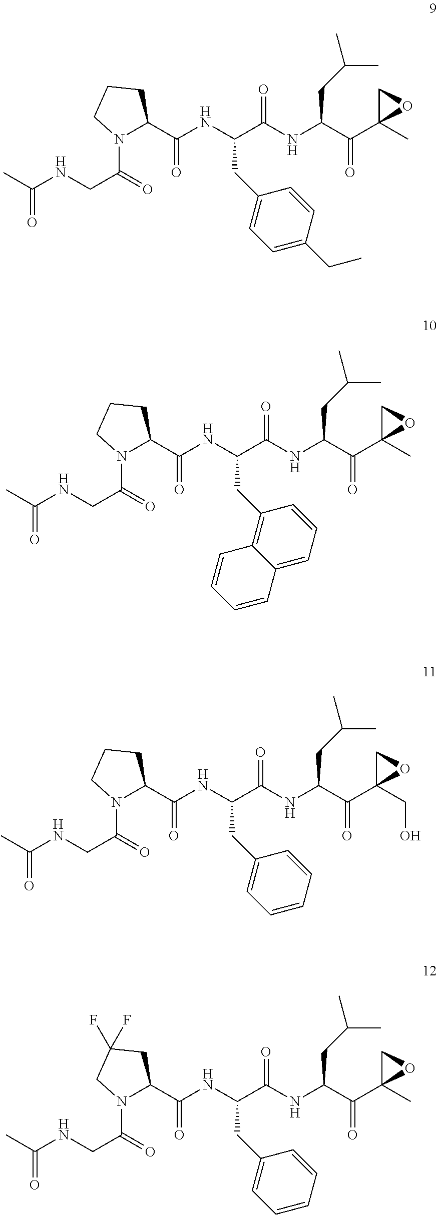

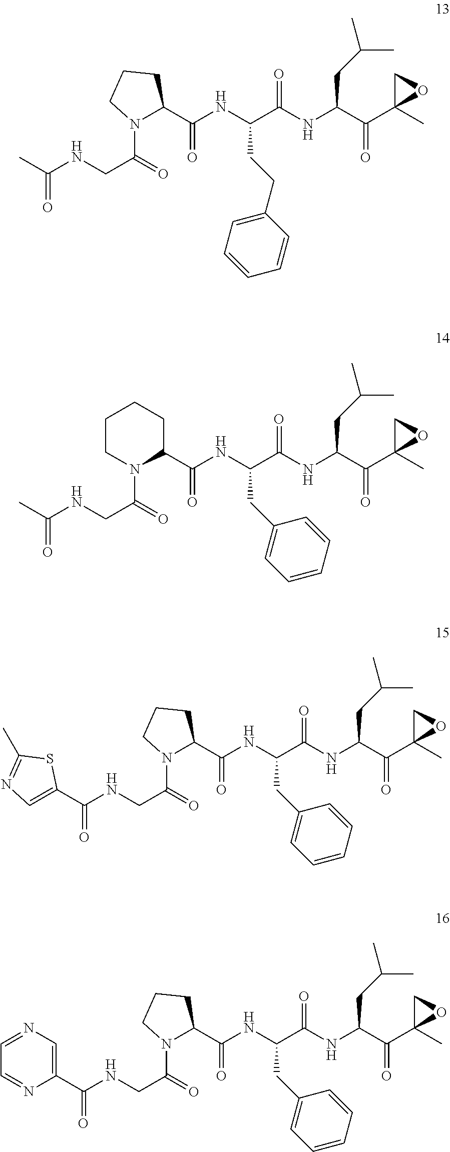

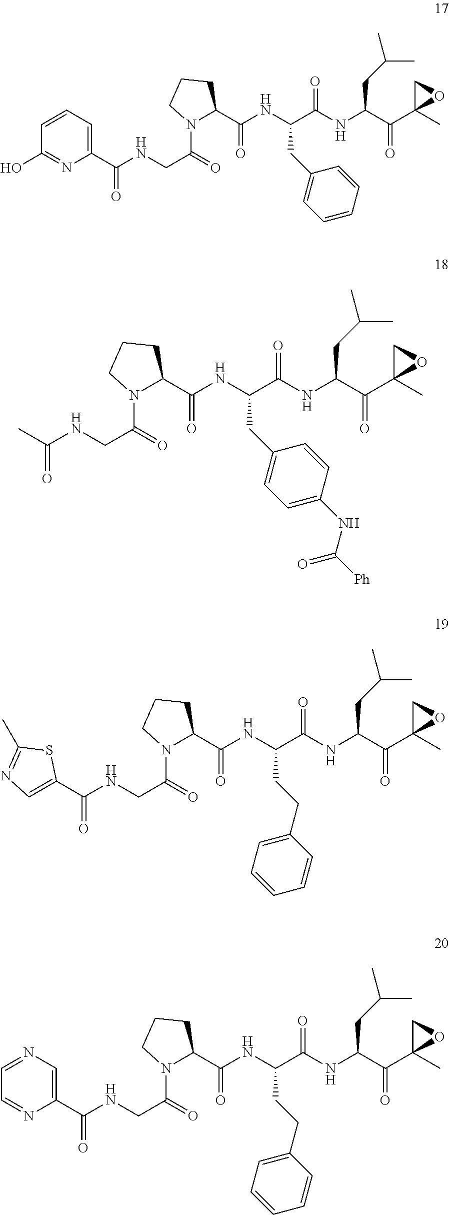

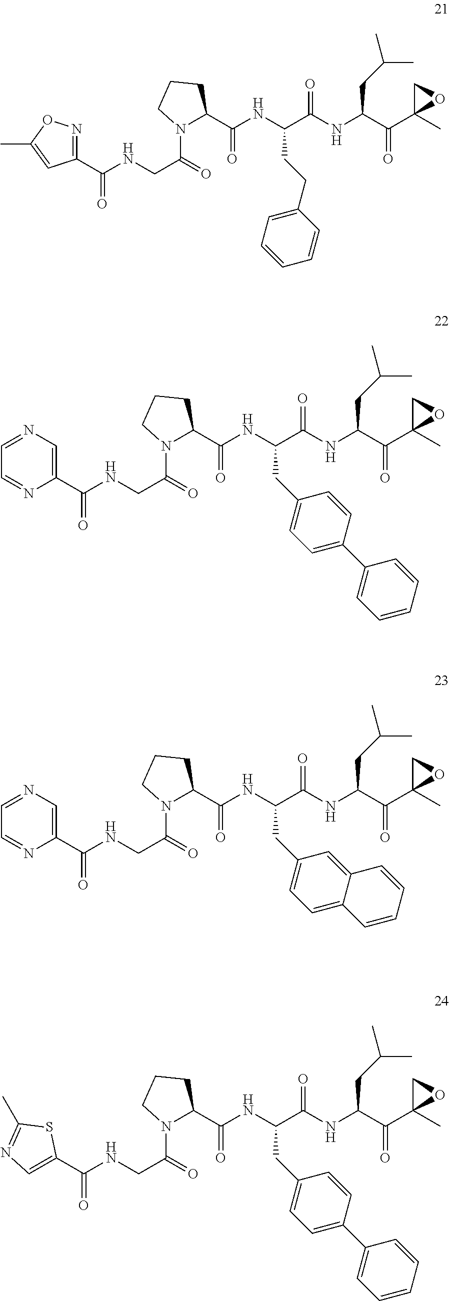

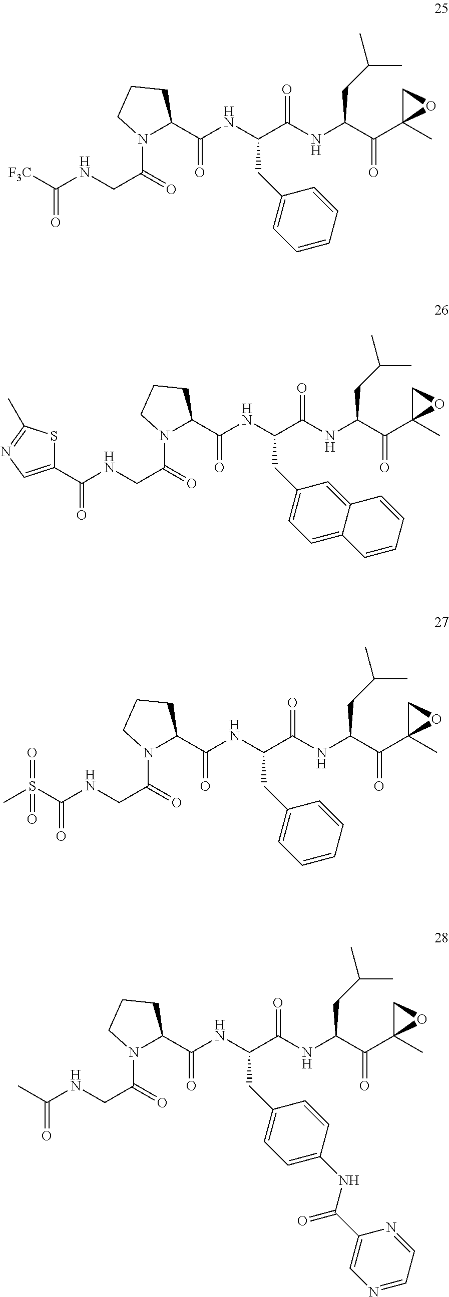

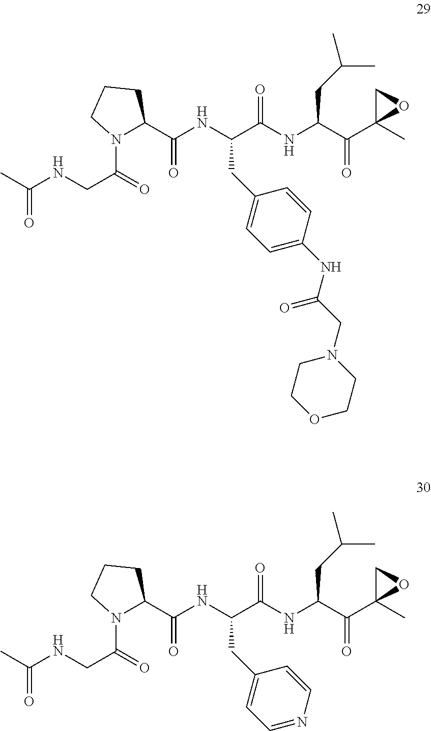

3. A pharmaceutical composition, comprising: the compound of claim 1, and a pharmaceutically-acceptable carrier.

4. A method of inhibiting a proteasome in a cell, comprising administering an effective amount of the compound of claim 1 to the cell.

5. The method of claim 4, wherein the method selectively inhibits the LMP2 of the cell.

6. The method of claim 4, wherein the cell is a cancer cell or a retinal pigment epithelial cell.

7. A method of treating a disease in a subject comprising administering an effective amount of a pharmaceutical composition containing the compound of claim 1 to the subject.

8. The method of claim 7, wherein the subject is in need of treatment for cancer, neurodegenerative disease, age related macular degeneration, or autoimmune disease.

9. The method of claim 8, wherein the subject is in need of treatment for Alzheimer's Disease.

10. The method of claim 9, wherein the administering results in memory function.

11. The method of claim 8, wherein the subject is in need of treatment for Multiple Myeloma.

12. The method of claim 11, wherein the Multiple Myeloma is resistant to carflizomib and/or bortezomib.
